SEQUENCE LISTING (1 sequence)

SEQ ID NO: 1 - protein, 221 aa, Artificial Sequence
  anti-mouse ICAM-1 scFv
HWVRQAPGKGLEWVSGISGSGRTTYYADSVKGRSSISRDNSKNTVTLELNSLRADDTAVYHCAKGGDSGRWDEWYFDLWGRGTLVTVSSGEGKSSGSGSESKASDVVMTQTPSSLSASVGDRVTITCRASQSISNYLNWYQQKPGKAPKLLIYAASSLQSGVPSRFSGSGSGTDFTLTISSLQPEDFATYYCQQSYSTPLTFGGGTKVDIKVEASHHHHHH

(19) United States
(12) Patent Application Publication
(10) Pub. No.: US 2018/0177724 A1
(43) Pub. Date: Jun. 28, 2018

(54) SITE-TARGETED NANO-LIPOSOMAL NITROGLYCERIN THERAPEUTICS

(71) Applicant: The Regents of the University of California, Oakland, CA (US)

(72) Inventors: Kaustabh Ghosh, Riverside, CA (US); Sharad Gupta, Riverside, CA (US); Soroush Ardekani, Riverside, CA (US); Umar Mohideen, Riverside, CA (US)

(21) Appl. No.: 15/738,976

(22) PCT Filed: Jul. 1, 2016
(86) PCT No.: PCT/US16/40698

§ 371 Date: Dec. 21, 2017

Related U.S. Application Data
(60) Provisional application No. 62/188,474, filed on Jul. 2, 2015.
(60) Provisional application No. 62/253,304, filed on Nov. 10, 2015.

(51) Int. Cl. A61K 9/127; A61K 31/21; A61K 47/69

(52) U.S. Cl. CPC A61K 9/127; A61K 47/6913; A61K 31/21

(57) ABSTRACT

The disclosure provides for nanoliposomal formulations comprising nitroglycerin, methods of making the formulations, and methods of use thereof.

### CROSS REFERENCE TO RELATED APPLICATIONS

The application claims priority under 35 U.S.C. § 119 to U.S. Provisional Application Ser. No. 62/188,474, filed Jul. 2, 2015 and U.S. Provisional Application Ser. No. 62/253,304, filed Nov. 10, 2015, the disclosures of which are incorporated herein by reference.

### TECHNICAL FIELD

The disclosure provides for nanoliposomal formulations comprising nitroglycerin, methods of making the formulations, and methods of use thereof.

### BACKGROUND

Nitroglycerin (NTG) markedly enhances nitric oxide (NO) bioavailability. However, its ability to mimic the anti-inflammatory properties of NO remains unknown.

### SUMMARY

The disclosure provides for nanoliposomal NTG formulations (NTG-NL) that exhibit superior anti-inflammatory effects than free NTG. Moreover, the modified NTG-NL formulations were also shown to exert positive effects on mitochondrial superoxide production and loss of arterial vasorelaxation associated with high NTG doses. The NTG-NL formulations of the disclosure significantly inhibited human monocyte adhesion to NO-deficient human microvascular endothelial cells (ECs) in vitro (EC₅₀=0.64 uM) through an increase in endothelial nitric oxide (NO) and decrease in endothelial intercellular cell adhesion molecule (ICAM-1) clustering, as determined by NO analyzer, microfluorimetry, and immunofluorescence staining. The NTG-NL formulations of the disclosure were formulated by encapsulating NTG within unilamellar lipid nanoparticles (e.g., DPhPC, POPC, Cholesterol, DHPE-Texas Red at molar ratio of 6:2:2:0.2). These nanoparticles were ˜150 nm in diameter, and readily taken up by ECs, as determined by dynamic light scattering and quantitative fluorescence microscopy, respectively. More importantly, the NTG-NL formulations of the disclosure produced a 70-fold increase in NTG therapeutic efficacy when compared with free NTG while preventing excessive mitochondrial superoxide production and loss of arterial vasorelaxation associated with high NTG doses.

In a particular embodiment, the disclosure provides for a nitroglycerin-nanoliposome (NTG-NL) formulation comprising: nitroglycerin incorporated in nanoliposomes made from a plurality of lipids, wherein the nanoliposomes have a diameter between 10 and 500 nm or any diameter therebetween (e.g., 20-400, 50-300, 60-250, 100-200, 120-180, 140-160 nm etc.). In a further embodiment, the nanoliposomes are unilamellar liposomes or micelles. In anothermebodiment, the nanoliposomes are multilamellar liposomes. In another embodiment, the plurality of lipids comprise phospholipids selected from phosphatidylcholine, phosphatidic acid, phosphatidylethanolamine, phosphatidylglycerol, phosphatidylserine, lysophosphatidylcholine, and/or any derivative thereof. In yet another embodiment, the phospholipid derivatives are selected from 1,2-di-(3,7,11,15-tetramethylhexadecanoyl)-sn-glycero-3-phosphocholine, 1,2-didecanoyl-sn-glycero-3-phosphocholine, 1,2-dierucoyl-sn-glycero-3-phosphate, 1,2-dierucoyl-sn-glycero-3-phosphocholine, 1,2-dierucoyl-sn-glycero-3-phosphoethanolamine, 1,2-dilinoleoyl-sn-glycero-3-phosphocholine, 1,2-dilauroyl-sn-glycero-3-phosphate, 1,2-dilauroyl-sn-glycero-3-phosphocholine, 1,2-dilauroyl-sn-glycero-3-phosphoethanolamine, 1,2-dilauroyl-sn-glycero-3-phospho-(1′-rac-glycerol), 1,2-dimyristoyl-sn-glycero-3-phosphate, 1,2-dimyristoyl-sn-glycero-3-phosphocholine, 1,2-dimyristoyl-sn-glycero-3-phosphoethanolamine, 1,2-dimyristoyl-sn-glycero-3-phosphoglycerol, 1,2-dimyristoyl-sn-glycero-3-phosphoserine, 1,2-dioleoyl-sn-glycero-3-phosphate, 1,2-dioleoyl-sn-glycero-3-phosphocholine, 1,2-dioleoyl-sn-glycero-3-phosphoethanolamine, L-alpha-phosphatidyl-DL-glycerol, 1,2-dioleoyl-sn-glycero-3-phosphoserine, 1,2-dipalmitoyl-sn-glycero-3-phosphate, 1,2-dipalmitoyl-sn-glycero-3-phosphocholine, 1,2-dipalmitoyl-sn-glycero-3-phosphoethanolamine, 1,2-dipalmitoyl-sn-glycero-3-phosphoglycerol, 1,2-dipalmitoyl-sn-glycero-3-phosphoserine, 1,2-distearoyl-sn-glycero-3-phosphate, 1,2-distearoyl-sn-glycero-3-phosphocholine, 1,2-distearoyl-sn-glycero-3-phosphoethanolamine, 1,2-distearoyl-sn-glycero-3-phosphoglycerol, egg sphingomyelin, egg-PC, hydrogenated Egg PC, hydrogenated Soy PC, 1-myristoyl-sn-glycero-3-phosphocholine, 1-palmitoyl-sn-glycero-3-phosphocholine, 1-stearoyl-sn-glycero-3-phosphocholine, 1-myristoyl-2-palmitoyl-sn-glycero 3-phosphocholine, 1-myristoyl-2-stearoyl-sn-glycero-3-phosphocholine, 1-palmitoyl-2-myristoyl-sn-glycero-3-phosphocholine, 1-palmitoyl-2-oleoyl-sn-glycero-3-phosphocholine, 1-palmitoyl-2-oleoyl-sn-glycero-3-phosphoethanolamine, 1-palmitoyl-2-oleoyl-sn-glycero-3-phosphoglycerol, 1-palmitoyl-2-stearoyl-sn-glycero-3-phosphocholine, 1-stearoyl-2-myristoyl-sn-glycero-3-phosphocholine, 1-stearoyl-2-oleoyl-sn-glycero-3-phosphocholine, and/or 1-stearoyl-2-palmitoyl-sn-glycero-3-phosphocholine. In a further embodiment, the liposomes further comprise cholesterol. In yet another embodiment, the liposomes further comprise polyethylene glycol. In a particular embodiment, the liposomes further comprise one or more site-targeting moieties. Examples of site-targeting moieties include, but are not limited to, peptides, aptamers, antibodies, and antibody fragments (e.g., F(ab′)₂, Fab, and scFv).

In a certain embodiment, the disclosure provides for a NTG-NL formulation disclosed herein which is formulated for enteral delivery, parenteral delivery, topical delivery, or by inhalation.

In another embodiment, the nitroglycerin to nanoliposome ratio by weight for a NTG-NL formulation disclosed herein is from 1:20 to 20:1. In alternate embodiment, the nitroglycerin to nanoliposome ratio by weight for a NTG-NL formulation disclosed herein is from 1:10 to 10:1. In a particular embodiment, the nitroglycerin to nanoliposome ratio by weight for a NTG-NL formulation disclosed herein is from 1:10.

In a certain embodiment, the disclosure further provides a method for treating a disease or disorder associated with loss of endogenous vascular endothelial nitric oxide, increased expression of endothelial cell adhesion molecules (CAMs) (e.g. ICAM-1); or increased clustering of endothelial CAMs (e.g. ICAM-1), vascular inflammation, hyperpermeability, regression, or vasoconstriction in a subject comprising administering the NTG-NL formulation disclosed herein to the subject. Examples of such disease or disorders, include pulmonary arterial hypertension (PAH), atherosclerosis, diabetes (including, e.g., endothelial dysfunction and/or vascular inflammation associated with diabetes, such as diabetic retinopathy, nephropathy, neuropathy, and cardiovascular disease), asthma, chronic peptic ulcer, tuberculosis, rheumatoid arthritis, chronic periodontitis, ulcerative colitis, Crohn's disease, chronic sinusitis, and chronic active hepatitis. In a particular embodiment, a subject with pulmonary arterial hypertension (PAH) or atherosclerosis can be treated by administering a nanoliposomal formulation disclosed herein.

In a certain embodiment, the disclosure provides for a NTG-NL formulation disclosed herein, wherein the formulation comprises: nitroglycerin incorporated in nanoliposomes made from a plurality of lipids, wherein the nanoliposomes have a diameter between 10 to 500 nm and wherein at a portion of the plurality of lipids are conjugated with polyethylene glycol (PEG). In a further embodiment, at least a portion of the PEG-conjugated lipids further comprise maleimide groups. In another embodiment, at least a portion of the PEG-conjugated lipids further comprise site-targeting moieties. In yet another embodiment, the site-targeting moieties are conjugated to the PEG-conjugated lipids using maleimide-thiol reaction chemistry. Examples of site-targeting moieties include but are not limited to peptides, aptamers, antibodies, and antibody fragments (e.g., antibody fragments are selected from F(ab′)₂, Fab, and scFv). In a further embodiment, the NTG-NL formulation comprising is formulated for enteral delivery, parenteral delivery, topical delivery, or by inhalation. In yet a further embodiment, the NTG-NL formulation of the disclosure comprises nitroglycerin to nanoliposome by weight in a ratio from 1:20 to 20:1, 1:10 to 10:1, or 1:10. In a particular embodiment, the disclosure provides for treating a disease or disorder associated with vascular inflammation in a subject comprising administering the NTG-NL formulation described herein. Examples of such diseases or disorders includes pulmonary arterial hypertension (PAH), atherosclerosis, diabetes (including, e.g., endothelial dysfunction and/or vascular inflammation associated with diabetes, such as diabetic retinopathy, nephropathy, neuropathy, and cardiovascular disease), asthma, chronic peptic ulcer, tuberculosis, rheumatoid arthritis, chronic periodontitis, ulcerative colitis, Crohn's disease, chronic sinusitis, and chronic active hepatitis. In a certain embodiment, the disease or disorder is pulmonary arterial hypertension (PAH) or atherosclerosis.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-C shows that NTG exerts anti-inflammatory effects on activated ECs. NTG produces dose-dependent inhibition of U937 monocytic cell adhesion to L-NIO-treated ECs, as shown in the fluorescence images (A) and quantified in the bar graph (n=10 fields of view) (B). (C) Similar anti-inflammatory effect of NTG was observed on TNFα-treated ECs (n=10 fields of view). **, p<0.01; ***, p<0.001. Data are expressed as mean±SEM. Scale bar: 200 μm.

FIG. 2A-C demonstrates that NTG enhances endothelial NO production. (A) Addition of NTG (5 μM) to L-NIO-treated ECs restores endothelial NO production, as determined by NOA measurement of extracellular NO (n=4 replicates per condition). Data normalized with respected to untreated control condition (con). (B) Fluorescent images of ECs labeled with NO-sensitive dye (DAF-FM diacetate) and subsequent image analysis (bar graph; n=at least 30 cells) confirms that NTG-mediated increase in NO results from greater endothelial NO synthesis. Scale bar: 25 μm. (C) Staining of U937 cell-EC co-cultures with anti-ICAM-1 and phalloidin (to label actin), coupled with fluorescent intensity measurement (bar graph; n=at least 20 cells), indicates that the anti-inflammatory effect of NTG (5 μM) correlates strongly with its ability to suppress ICAM-1 clustering induced by NO deficiency (L-NIO treatment). **, p<0.01; ***, p<0.001. Scale bar: 25 μm. Data are expressed as mean±SEM.

FIG. 3A-E presents the synthesis and physicochemical characterization of nanoliposomal NTG (NTG-NL). (A) Schematic of NTG molecule depicting the hydrophobic (—CH₂—CH₂—) and hydrophilic (ONOO⁻) groups. (B) For incorporation within nanoliposomes (NLs), NTG was mixed with four lipids viz. DPhPC, POPC, Cholesterol, and DHPE-Texas Red, which self-assemble to form nanoliposomes in an aqueous solution. (C) ESI-Mass Spec. measurements reveal that, at 10% w/w initial NTG loading, nanoliposomes exhibit successful NTG incorporation (˜37% incorporation efficiency). (D) Dynamic Light Scattering (DLS) analysis reveals that both blank and NTG-loaded NLs exhibit similar diameter (˜155 nm). (E) Size distribution of NL was independently confirmed using environmental scanning electron microscope (ESEM). Scale bar: 200 nm. Data are expressed as mean±Std Dev.

FIG. 4A-B shows the cellular uptake of nanoliposomes. (A) Spectrophotometer measurements of internalized fluorescently-labeled nanoliposomes (NL) reveal time-dependent uptake of NLs by cultured ECs (n=5 replicates per condition). (B) Fluorescent images of internalized NLs indicate strong colocalization with LysoTracker Red™-labeled endocytic vesicles (lysosomes and endosomes). Scale bar: 10 μm. ***, p<0.001. Data are expressed as mean±SEM.

FIG. 5A-B demonstrates that NTG-NL exerts superior anti-inflammatory effects. (A) Blank NLs are inert to ECs as cells treated with blank NLs exhibit neither an increase in U937 cell adhesion (with respect to control) nor a decrease in LNIO-induced U937 cell adhesion (n=10 fields of view). (B) Addition of 5 μg/ml NTG-NL 0.07 μM NTG) to L-NIO-treated ECs significantly inhibits U937 cell-EC adhesion, which is comparable to the inhibition produced by a 70-fold greater dose of free NTG (5 μM). Free NTG dose of 0.07 μM does not produce a significant anti-inflammatory effect. (n=10 fields of view). **, p<0.01; ***, p<0.001. Data are expressed as mean±SEM.

FIG. 6A-C shows that NTG-NL enhances endothelial NO production. (A, B) Immunofluorescent staining of ECs labeled with NO-sensitive dye (DAF-FM diacetate) and subsequent image analysis (bar graph; n=at least 30 cells) confirms that, like free NTG, NTG-NL enhances NO production in L-NIO-treated cells. (C) Immunofluorescent staining of U937 cell-EC co-cultures for ICAM-1 and subsequent fluorescent intensity measurement (bar graph; n=at least 20 cells) reveals that, like free NTG (5 μM), NTG-NL (5 μg/ml) suppresses ICAM-1 clustering induced by NO deficiency (L-NIO treatment). **, p<0.01; *^{**}, p<0.001. Data are expressed as mean±SEM.

FIG. 7A-D demonstrates that NTG-NL prevents Endothelial Superoxide Formation Associated with High NTG Dose. (A) Representative fluorescent images of MitoSOX™-labeled ECs and (B) subsequent quantification (bar graph; n=at least 30 cells) indicate that addition of 20-fold higher dose (100 μM) of free NTG to L-NIO-treated ECs significantly increases mitochondrial superoxide formation while similar increase in NTG-NL dose produces no effect. Scale bar: 100 μm. Therapeutic dose is indicated in bold. (C) The high dose of free NTG fails to suppress U937 monocytic cell adhesion to L-NIO-treated ECs (n=10 fields of view). (D) NTG-NL continues to exert potent anti-inflammatory effect on L-NIO-treated ECs at the 20-fold higher dose (n=10 fields of view). ***, p<0.001. Data are expressed as mean±SEM.

FIG. 8A-B provides for the measurement of arterial vasorelaxation in response to free NTG and NTG-NL treatment. (A) Pulmonary arterial rings pretreated with 100 μM free NTG exhibit impaired responsiveness to acute NTG treatment, as indicated by a significant rightward shift in NTG dose-relaxation curve (n≥eight arterial rings). Therapeutic dose is indicated in bold. (B) In contrast, arterial rings pretreated with a similar 20-fold higher NTG-NL dose exhibit normal relaxation response to acute NTG treatment. Data are expressed as mean±SEM.

FIG. 9 demonstrates that EC density and spreading are similar across various treatment conditions. Phase images of ECs treated without or with L-NIO±NTG or NTG-NL show that cell spreading is similar across all treatment conditions. Therefore, the difference in MitoSox® fluorescence intensity seen in FIG. 7A reflects the actual difference in mitochondrial superoxide production. The effective therapeutic NTG dose is indicated in bold. Scale bar=100 μm.

FIG. 10 demonstrates that NTG-NL Suppresses endothelial vascular inflammation after just 4 h treatment. EC monolayers were treated with L-NIO±NTG or NTG-NL for 4 hr prior to addition of fluorescently-labeled U937 monocyte cells. Quantification of adherent U937 cells (per mm2) on EC monolayers (n=10 fields of view) show that both NTG at 5 μM and NTG-NL at 5 μg/ml produce significant inhibition of U937 cell-EC adhesion within 4 hr of treatment. Further, while free NTG loses its therapeutic effect at a 20-fold higher dose of 100 μM, NTG-NL retains its immunosuppressive effects at a similar 20-fold greater dose (100 μg/mL). Thus, the anti-inflammatory effects of free NTG and NTG-NL observed after 4 h of treatment are consistent with those seen following overnight treatment. ***, p<0.001. The effective therapeutic NTG dose is indicated in bold. Data are expressed as mean±SEM.

FIG. 11A-C demonstrates NTG-NL-anti-ICAM-1 preferential targeting to inflamed retinal vessels in vivo. To enhance ICAM1 expression in mouse retinal vessels, angiopoietin-2 (Ang-2) was injected in one eye of adult mice. Ang-2 is known to enhance ICAM-1 expression in vascular endothelial cells (ECs). The other eye that received no Ang-2 served as control. To evaluate site-targeting capability of anti-ICAM1-conjugated nanoliposomes (NL-anti-ICAM1), these or unconjugated (control) nanoliposomes were administered (I.V.) into the angiopoietin-2-treated mice. To facilitate imaging, nanoliposomes were labeled with a red fluorescent dye. As shown in the fluorescent images above, NL-anti-ICAM-1 preferentially accumulated within ICAM-1-expressing retinal vessels but not in control eyes (A, B). Importantly, this preferential targeting was not seen with unconjugated nanoliposomes (C). Scale Bar: 50 μm.

FIG. 12A-B presents the synthesis and physicochemical characterization of scFv. (A) Schematic diagram describing the cloning strategy for constructing scFv fragments of ICAM-1 antibody into the pMopac vector. Variable domains of heavy chain and light chain were linked by a (Gly₄Ser)₃ linker and then incorporated with a histidine (HIS)₆ tag attached to a free cysteine at the end. (B) To determine whether the engineered scFv had binding affinity for ICAM-1-expressing ECs, pulmonary arterial EC (PAEC) monolayers were stimulated with TNF-α for 4 h in starvation media (2.5% FBS) to enhance ICAM-1 expression, followed by incubation with scFv at varying doses: 0.06 μM, 0.3 μM, and 1.5 μM for 20 min at 4° C. Next, EC were incubated with mouse anti-HIS antibody for 20 min at 4° C., followed by FITC-conjugated DyLight 488 anti-mouse IgG for an additional 20 min. Next, ECs were fixed with 1% paraformaldehyde detected by a Cell Lab Quanta SC flow cytometer, and analyzed by FlowJo. Flow cytometry plots indicate dose-dependent binding of scFv to ICAM-1-expressing ECs.

FIG. 13A-B presents the synthesis and physicochemical characterization of NL-scFv. (A) Schematic depicting the synthesis of scFv-modified nanoliposomes (NL-scFv) using five lipids viz. DSPE-PEG-Maleimide, DPhPC, POPC, Cholesterol, and DHPE-Texas Red at different compositions (20, 60, 20, and 0.2 mol %). The lipids were combined that self-assembled to form NL in an aqueous solution (H₂O). scFv fragments were conjugated on the surface of NL using maleimide-thiol reaction chemistry, followed by quenching of the remaining free maleimide groups with free cysteine (10 μM) to develop NL-scFv. (B) To detect conjugation of scFv to NL surface, NL-scFv was labeled with mouse anti-HIS antibody for 2 h at room temperature (RT) followed by FITC-conjugated DyLight 488 anti-mouse IgG for an additional 2 h at RT. scFv conjugation to NL surface was quantified using a Flexstation 2 fluorescent microplate reader (n=3 per condition). Representative line graph shows the linear correlation between the amount of scFv added to NL suspension (x-axis) and the amount conjugated to NL surface (fluorescence intensity; y-axis).

FIG. 14A-B shows NL-scFv undergo preferential uptake by inflamed (ICAM-1-expressing) human PAECs. (A) PAEC monolayers were first treated with TNF-α (10 ng/mL) before addition of Texas Red™-labeled NLs conjugated with different scFv doses viz. 0, 0.76, 1.9 and 3.8 μM for 30 min at 37° C. EC monolayers were then gently rinsed twice with PBS prior to fixation in 1% PFA. Fluorescent images of internalized NL-scFv show significant uptake by TNF-α-stimulated (inflamed) PAECs (TNF-α; 10 ng/mL) in a dose-dependent manner. (B) To quantify the extent of NL-scFv uptake at different doses of surface scFv, fluorescent images (8 per condition) of NL-scFv-treated ECs were acquired using an Leica Sp5 Confocal Microscope (Leica, Germany) and cell fluorescent intensities quantified in the bar graph (n≥20) using ImageJ software (NIH). Quantitative measurement of fluorescence intensity of NL-scFv-treated ECs revealed ˜6-fold greater binding of NL-scFv conjugates to TNF-α-stimulated PAECs compared to unstimulated ECs. Scale bar: 100 μm. **, p<0.01; ***, p<0.001.

FIG. 15 Shows the Kinetics of NTG Release from NTG-NL. To measure the release kinetics of incorporated NTG, 300 μg of NTG-NL was suspended in water and incubated at 37° C. for pre-determined time durations (n=3 per time point). At the end of each time point, NTG-NL was pelleted by ultracentrifugation at 60,000 rcf for 1.5 hr and pellets dissolved in 200 μl methanol for Electron Spray Ionization (ESI)-Mass Spectrometry (MS) measurement of residual NTG. The ESI-Mass Spec data demonstrates an initial rapid release of NTG, followed by a slower, more sustained release at longer intervals.

FIG. 16 shows NTG-NL-scFv exhibits potent anti-inflammatory effects. To examine the anti-inflammatory effects of NTG-NL-scFv, PAEC monolayers were first treated with TNF-α (10 ng/ml), followed by addition of NTG-NL or NTG-NL-scFv (5 μg/ml) for 4 hr. Next, fluorescently-labeled human U937 monocytic cells (130,000 cells/cm2) were added for 30 minutes at 37° C. Following two rinses with PBS, adherent monocytes were fixed with 1% PFA, imaged using Nikon Eclipse Ti microscope fitted with a Nikon DS-Qi1Mc camera, and counted using ImageJ (≥10 images per condition). Quantification of adherent U937 cells revealed that NTG-NL-scFv exhibits a ˜two-fold greater anti-inflammatory effect than non-modified NTG-NL. *, p<0.05 **, p<0.01; ***, p<0.001; ns, no significance.

FIG. 17 shows NL uptake by activated U937 monocytes. To measure NL uptake, U937 cells were first differentiated to activated macrophages by treatment with phorbolmyristate acetate (PMA; 100 ng/ml) for 48 hr, as commonly reported. Cells were then detached and incubated with fluorescently-labeled NL, NL-PEG, and NL-PEG-scFv at a dose of 400 μg/ml in suspension at 37° C. After 30 min, U937 cells were rinsed twice with PBS to remove non-internalized NLs, and fixed in 1% PFA. Following fixation, U937 cells were labeled with 300 nM DAPI (to visualize nucleus) for 5 min and rinsed twice. DAPI-labeled U937 cells were imaged using an Sp5 confocal microscope and then transferred to a black-walled 96-well plate (n=3) for NL fluorescence measurement using a Flexstation II 384 fluorescent plate reader. Representative fluorescent images show internalization of different NL formulations by activated U937 cells. Scale bar: 5 μm Fluorescence intensity analysis reveals that when compared with non-PEGylated NLs, PEG-modified NLs and NL-scFv undergo a significant decrease in uptake by activated U937 cells. **, p<0.01; ***, p<0.001.

FIG. 18 shows the amino acid sequence (SEQ ID NO:1) of mouse anti-ICAM-1 scFv that was generated by screening mouse ICAM-1 antigen against a proprietary naïve scFv phage library (Neoclone, WI, USA).

FIG. 19 shows binding of mouse scfv to inflamed (ICAM-1-expressing) mouse ECs. To determine whether the mouse scFv exhibits binding affinity to ICAM-1-expressing mouse ECs, EC monolayers were stimulated with TNF-α for 4 hr in starvation media (2.5% FBS) to enhance ICAM-1 expression, followed by incubation with scFv 5 μM for 20 min at 4° C. Next, EC were incubated with rabbit anti-FLAG antibody for 20 min at 4° C., followed by Texas Red™-conjugated 594 goat anti-rabbit IgG for an additional 20 min. Next, ECs were fixed with 1% paraformaldehyde detected by a Novocyte flow cytometer, and analyzed by FlowJo.

FIG. 20 shows that anti-ICAM-1 scFv demonstrates function-blocking effects through inhibition of monocyte-EC adhesion. To examine the function-blocking effects of anti-ICAM-1 scFv, mouse EC monolayers were first treated with TNF-α (10 ng/ml), followed by addition of scFv (5 μM) for 30 min. Next, fluorescently-labeled mouse monocytes were added for 30 minutes at 37° C. Following two rinses with PBS, adherent monocytes were fixed with 1% PFA, imaged using Nikon Eclipse Ti microscope fitted with a Nikon DS-Qi1Mc camera, and counted using ImageJ (10 images per condition). Quantification of adherent monocytes revealed that mouse scFv significantly reduced monocyte adhesion ***, p<0.001. Scale bar: 200 μm

### DETAILED DESCRIPTION

As used herein and in the appended claims, the singular forms “a,” “an,” and “the” include plural referents unless the context clearly dictates otherwise. Thus, for example, reference to “a liposome” includes a plurality of such liposome and reference to “the agent” includes reference to one or more agents, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Loss of endothelium-derived nitric oxide (NO), which prevents leukocyte-endothelial cell (EC) adhesion, is strongly implicated in chronic vascular inflammation associated with debilitating cardiovascular conditions such as pulmonary arterial hypertension (PAH), atherosclerosis, and diabetes. Administration of nitrates/nitrites, which rapidly produce NO, is thus being explored as anti-inflammatory therapy. Since organic nitrates exert superior NO-dependent vasodilatory effects when compared with inorganic nitrates/nitrites, they likely also exhibit more potent anti-inflammatory effects.

Of the clinically used organic nitrates, nitroglycerin (NTG) holds particular promise because, in addition to spontaneously producing NO via mitochondrial aldehyde dehydrogenase (ALDH-2), it also activates endothelial NO synthase (eNOS), the key NO-producing enzyme in ECs that is impaired in inflammatory cardiovascular conditions. However, despite its potential anti-inflammatory effects, NTG presents a conundrum as long-term clinical use of current NTG formulations causes adverse effects such as impaired vasorelaxation in response to acute NTG treatment (NTG tolerance, which limit its therapeutic efficacy. Thus, new NTG delivery approaches are required to fully leverage the therapeutic potential of NTG.

Disclosed herein is NTG nanoformulation which can suppress endothelial cell (EC) activation during inflammation (e.g., vascular inflammation), and simultaneously amplify its anti-inflammatory effects. Moreover, the NTG nanoformulation disclosed herein can ameliorate adverse effects associated with high-dose NTG administration. The findings presented herein reveal that NTG significantly inhibits human monocyte adhesion to NO-deficient human microvascular ECs in vitro (EC₅₀=0.64 uM) through an increase in endothelial NO and decrease in endothelial ICAM-1 clustering, as determined by NO analyzer, microfluorimetry, and immunofluorescence staining. In a particular embodiment, nanoliposomal NTG (NTG-NL) was formulated by encapsulating NTG within unilamellar lipid nanoparticles (e.g., DPhPC, POPC, Cholesterol, DHPE-Texas Red at molar ratio of 6:2:2:0.2). The nanoparticles were 150 nm in diameter and readily taken up by ECs, as determined by dynamic light scattering and quantitative fluorescence microscopy, respectively. More importantly, NTG-NL produced a 70-fold increase NTG therapeutic efficacy when compared with free NTG while preventing excessive mitochondrial superoxide production and loss of sheep arterial vasorelaxation associated with high NTG doses.

The disclosure provides for nitroglycerin-nanoliposomal (NTG-NL) formulations that exert superior therapeutic effects and further their use in vascular normalization therapies. The NTG nanoformulations described herein provide for effective NTG delivery that exhibits anti-inflammatory effects while preventing excessive mitochondrial superoxide production and impaired vasorelaxation associated with high-dose NTG treatment. Thus, by not having the same adverse effects, the NTG nanoformulations of the disclosure are noticeably superior to conventional NTG therapy. NTG is the most commonly used organic nitrate in the clinic where it is intended to mimic the vasodilatory effects of endothelium-derived NO. When compared with inorganic nitrites/nitrates, NTG produces a significantly greater and rapid yield of NO, which explains its superior vasodilatory properties. Since endothelial NO also exhibits potent anti-inflammatory effects, it was determined herein whether NTG can suppress leukocyte-EC adhesion.

The findings presented herein reveal that NTG strongly inhibited monocyte adhesion to NO-deficient ECs. Further, NTG treatment produced significant inhibition of ICAM-1 clustering on EC surface. Although inorganic nitrites have been shown to exert anti-inflammatory effects, the studies indicate herein that NTG also exhibits a similar effect. NTG activates eNOS, the major NO-producing enzyme in ECs that is impaired in inflammatory conditions such as PAH and diabetes. Further, NTG mimics the anti-coagulating properties of NO to prevent inflammation-associated hypercoagulopathy (e.g., vascular inflammation-associated hypercoagulopathy). Thus, the NTG-NL formulations disclosed herein can be used with any disease or disorder associated with inflammation (e.g., vascular inflammation), such as asthma, chronic peptic ulcer, tuberculosis, rheumatoid arthritis, chronic periodontitis, ulcerative colitis, Crohn's disease, chronic sinusitis, chronic active hepatitis, pulmonary arterial hypertension (PAH), diabetic vascular complications (e.g., retinopthy, nephropathy, neuropathy), and cardiovascular diseases.

Despite widespread use of NTG as a vasodilatory drug and its promising anti-inflammatory potential, tolerance and cross-tolerance (endothelial dysfunction) associated with large clinical doses of current NTG formulations limit its efficacy. These adverse effects of high NTG doses, presumably given to offset the rapid (within 15-30 min) clearance of NTG from bloodstream, are mediated by excessive formation of mitochondrial ROS that irreversibly inhibits ALDH-2, thereby impairing NTG bioconversion to NO and, consequently, reducing NTG sensitivity.

To improve the benefit/risk profile of NTG therapies, the principles of nanotechnology were leverage to develop an innovative NTG-NL formulation that demonstrated a remarkable 70-fold increase in therapeutic (anti-inflammatory) effect. The significant enhancement in NTG-NL bioactivity and resultant lowering of the effective therapeutic dose meant that, unlike free NTG, NTG-NL did not elicit an increase in mitochondrial ROS production (tolerance) even at very high (20-fold greater) doses. This observation is supported by findings that, in contrast to free NTG, NTG-NL treatment at high dose did not exhibit any loss of anti-inflammatory effect or produce rightward shift in dose-relaxation response of isolated pulmonary arteries.

The size (˜150 nm diameter) of the NTG-NL formulations disclosed herein is suitable for use as long-circulating nanoparticles. It is further contemplated herein, that the nanoliposomal NTG formulations can be adapted for site-targeting, by tethering targeting moieties (peptides, aptamers, antibodies, antibody fragments, sugar or glycolipids) on the nanoparticle surface which can guide the nanotherapeutic selectively to desired vascular sites, thereby facilitating local drug delivery and therapeutic effects. By addressing the adverse effects associated with conventional high-dose NTG formulations, the nanoliposomal NTG formulation (NTG-NL) disclosed herein can leverage the anti-inflammatory and vasodilatory properties of NTG for superior management of PAH that is characterized by both severe vasoconstriction and chronic pulmonary arterial vascular inflammation. Various linking groups can be used for joining the lipid chains of the liposome to a targeting ligand (Mannino et al., Bio Techniques 6(7):682, 1988, incorporated by reference). For example, various reactive groups can be employed to tether targeting groups to the lipids making up the nanoliposomes disclosed herein, such as sulfhydryl-reactive groups, maleimides, haloacetyls, pyridyldisulfides, thiosulfonates, and vinylsulfones; carboxyl-to-amine reactive groups, such as carbodiimides (e.g., EDC); amine-reactive groups, such as NHS esters, imidoesters, pentafluorophenyl esters, hydroxylmethyl phosphine; aldehyde-reactive groups, such as hydrazides, and alkoxyamines; photoreactive groups, such as diazinine, and aryl azide; and hydroxyl (nonaqueous)-reactive groups, such as isocyanates.

The compounds bound to the surface of the targeted delivery system may vary from small haptens of from about 125-200 molecular weight to much larger antigens with molecular weights of at least about 6 KD, but generally of less than 10⁶ KD. Proteinaceous ligand and receptors are of particular interest. Since the composition incorporated in the liposome may be indiscriminate with respect to cell type in its action, a targeted delivery system offers a significant improvement over randomly injecting non-specific liposomes. A number of procedures can be used to covalently attach either polyclonal or monoclonal antibodies to a liposome bilayer. Antibody-targeted liposomes can include monoclonal or polyclonal antibodies or fragments thereof such as scFV, Fab, or F(ab′)₂, as long as they bind efficiently to the antigenic epitope on the target cells. Particularly advantageous targets for selective delivery of the NTG-NL formulation of the disclosure include cell surface proteins that are typically expressed on endothelial cells, including, but not limited to Tie-2 receptors, endothelial CAMs (e.g. ICAM-1, E-selectins, and VCAM-1).

By addressing the adverse effects associated with conventional high-dose NTG formulations, the nanoliposomal NTG formulation (NTG-NL) disclosed herein can leverage the anti-inflammatory and vasodilatory properties of NTG for superior management of PAH that is characterized by both severe vasoconstriction and chronic pulmonary arterial inflammation.

In a certain embodiment, a NTG-NL formulation disclosed herein can be administered directly or as a part of a composition. In other embodiments, the composition could be formulated as a pharmaceutically acceptable composition for administration to a subject. In another embodiment, a compound disclosed herein can be a part of a pharmaceutical composition which includes one or more pharmaceutically acceptable carriers. Pharmaceutically acceptable carriers include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy to administer by a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g. a compound disclosed herein, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, one or more NTG-NL formulations of the disclosure are prepared with carriers that will protect the compound against rapid elimination from the body, such as a controlled release formulation, including use of polyethylene glycol, implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc.

The data obtained from the cell culture assays and animal studies can be used in formulating a range of dosage for use in humans. The dosage of nitroglycerin lies preferably within a range of circulating concentrations that include the EC₅₀ with little or no toxicity. The dosage may vary within this range depending upon the dosage form employed and the route of administration utilized. For nitroglycerin used in the method of the disclosure, the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

NTG-NL formulations disclosed herein, including pharmaceutical compositions comprising said formulations, can be used to treat a disorder or disease associated with inflammation (e.g., vascular inflammation) in a subject. Examples of such disorders or diseases which can be treated include pulmonary arterial hypertension (PAH), atherosclerosis, diabetes (i.e., endothelial dysfunction and/or vascular inflammation associated with diabetes, such as diabetic retinopathy, nephropathy, neuropathy, and cardiovascular disease), asthma, chronic peptic ulcer, tuberculosis, rheumatoid arthritis, chronic periodontitis, ulcerative colitis, Crohn's disease, chronic sinusitis, and chronic active hepatitis.

In another embodiment, a method of treating pulmonary arterial hypertension or atherosclerosis in a subject comprises administering to a subject in need of such treatment a therapeutically effective amount of a pharmaceutical composition comprising a NTG-NL formulation disclosed herein and a pharmaceutically acceptable carrier.

The working examples below are provided to illustrate, not limit, the invention. Various parameters of the scientific methods employed in these examples are described in detail below and provide guidance for practicing the invention in general.

### Examples

Cell Culture.

Human microvascular endothelial cells (HMEC-1) were purchased from the Center for Disease Control (CDC) and cultured on gelatin-coated tissue culture dishes in growth medium composed of MCDB-131 (VWR International, USA) supplemented with 10% FBS (Fisherbrand, USA), 2 mM L-Glutamine (Invitrogen, USA), 1× antimycotic/antibiotic mixture (Life Technologies, USA), 10 ng/mL huEGF (Millipore, USA) and 1 μg/mL Hydrocortizone (Sigma Aldrich, USA). Human U937 monocyte cells were purchased from ATCC (Manassas, Va., USA) and cultured in suspension in growth medium composed of RPMI 1640 (Fisherbrand, USA) supplemented with 2 mM L-Glutamine (Invitrogen), 10 mM HEPES (Fisherbrand, USA), 10% FBS (Fisherbrand), antimycotic/antibiotic mixture (Life Technologies, USA), 1 mM sodium pyruvate (Life Technologies, USA) and 4.5 mg/mL glucose (Sigma Aldrich, USA).

Nanoparticle (NP) Formulation.

To synthesize NTG-loaded nanoliposomes (NTG-NL), four lipid molecules viz. 1,2-di-(3,7,11,15-tetramethylhexadecanoyl)-sn-glycero-3-phosphocholine (DPhPC; Avanti Lipids, USA), 1-hexadecanoyl-2-(9Z-octadecenoyl)-sn-glycero-3-phosphocholine (POPC; Avanti Lipids, USA), Cholesterol (Sigma Aldrich, USA), and 1,2-dihexadecanoyl-sn-glycero-3-phosphoethanolamine-triethylammonium salt (Texas Red-DHPE; Invitrogen, USA) were dissolved in chloroform at a molar ratio of 6:2:2:0.2, respectively, and purged with Nitrogen (N₂) to evaporate the chloroform. The resulting lipid cake was placed under vacuum for at least two hours prior to rehydration in aqueous NTG (5, 10 and 25% w/w of total lipid; Cerilliant, USA) or Fluorescein (1 mM; Sigma Aldrich, USA) solution to obtain a final 1 mg/mL drug- or dye-loaded liposome suspension. To obtain NTG-NLs, these liposome suspensions underwent five freeze-thaw cycles in liquid N₂ followed by eight extrusion cycles through a 100 nm polycarbonate membrane filter (Avanti Lipids, USA). Unincorporated NTG or fluorescein was discarded by spinning down NTG-NLs for two hours at 60,000 rcf using a refrigerated ultracentrifuge (Beckman Coulter, USA) and decanting the supernatant. The final NTG-NL pellet was suspended at 1 mg/mL in water and stored at 4° C. until use.

NTG Incorporation Efficiency.

To determine NTG incorporation efficiency within polymeric NPs and NTG-NLs, pellets of polymeric NP (1 mg) or NTG-NL (200 μg) were dissolved in 100% methanol and analyzed using electron spray ionization-mass spectroscopy (ESI-MS; Agilent Technologies). NTG (MW 227.1 Da) was ionized by trifluoroacetic acid (MW: 112.9 Da) and the signature NTG mass/charge spectrum peak was detected at 339.9 mass/charge (m/z; charge z=1 coulomb). Area under the NTG peak was measured for both the initial and incorporated NTG and their ratio was calculated to determine % NTG incorporation efficiency.

Nanoliposome Size and Morphology Characterization:

Blank NL and NTG-NL suspensions were prepared at 0.5 mg/mL in distilled water and size distribution measured by dynamic light scattering (DLS) using a Delsa Nano C Particle Analyzer (Beckman Coulter, USA). Microsoft Excel® and Origin Pro software were used to acquire and analyze the data. NL morphology was analyzed using scanning electron microscopy (SEM; FEI NNS450) operated in high vacuum mode. For SEM samples, NLs were fixed in 2.5% glutaraldehyde (Electron Microscopy Sciences, USA) for 2 h at 4° C. After fixation, 10 uL of NLs were added to Poly-L-Lysine (Sigma-Aldrich, USA)-coated 12 mm glass coverslips, allowed to settle for 5 minutes, and subjected to critical-point drying in liquid CO₂ (Critical-point-dryer Balzers CPD0202). Samples were then sputter-coated with chromium for 30 sec and analyzed using the SEM instrument described above.

Nanoliposome (NL) Uptake.

To determine the rate of NL uptake by ECs, Texas Red®-labeled NLs were diluted in EC culture medium at 5 μg/mL and added to ECs for 5, 15, 30 or 60 min at 37° C. To quantify the extent of NL uptake at different doses, Texas Red®-labeled NLs at 5, 10, 50 or 100 μg/mL were added to EC monolayer for 30 min at 37° C. After treatment, non-internalized NLs were removed by rinsing ECs twice with PBS, following by culturing ECs in phenol-red free MEM media (Life Technologies, USA) supplemented with 2 mM L-Glutamine and 10% FBS and measurement of fluorescence intensity by Wallac 1420 Victor2 fluorescent microplate plate reader (Perkin Elmer, USA). To determine % NL uptake, fluorescence intensity of NL-treated ECs was divided by the intensity obtained from unrinsed samples. Further, to determine whether NTG-NLs were successfully endocytosed, ECs treated with fluorescein-incorporated NLs were stained with LysoTracker® Deep Red (Invitrogen, USA) to label acidic organelles (lysosomes and endosomes).

Monocyte Adhesion Assay.

To examine the effects of NTG on monocyte-EC adhesion, confluent EC monolayers were either serum-starved (MCDB-131, 2.5% FBS, and 1× antibiotic/antimycotic supplement) overnight and treated with 10 ng/mL TNF-α (eBiosciecnes, USA) for 4 h or treated for 4 h or overnight with 5 mM N⁵-(1-iminoethyl-L-ornithine) (L-NIO; selective eNOS inhibitor; Cayman Chemical, MI, USA)±varying doses of NTG (0, 0.07, 0.2, 1 or 5 μM; Cambridge Isotope, MA) in regular medium, followed by addition of fluorescently-labeled U937 monocyte cells at a density of 130,000 cells/cm′. After 30 min of monocyte-EC interaction at 37° C., monocyte suspension was removed and the EC monolayers gently rinsed twice with PBS (to remove unbound monocytes) prior to fixation in 1% paraformaldehyde (PFA; Electron Microscopy Sciences, USA). Fluorescent images (10 per condition) of labeled U937 cells were then acquired using a Nikon Eclipse Ti microscope (Nikon, Japan) fitted with a Nikon Digital Sight DS-Qi1Mc camera and the number of adherent monocytes was counted using ImageJ software (NIH). For experiments involving NLs, ECs were incubated with 5 μg/mL of blank or 10% (w/w) NTG-loaded nanoparticles (NTG-NL) for 30 minutes at 37° C., rinsed with PBS to remove excess NLs, and cultured overnight prior to addition of fluorescently-labeled U937 cells (as described above).

Measurement of Endothelial Cell (EC)-Derived NO.

Two independent methods were used to measure the amount of NO produced by ECs. In the first method, ECs were treated overnight with L-NIO (5 mM)±NTG (5 μM) or NTG-NL (10 μg/mL), followed by incubation with a NO-sensitive fluorescent dye DAF-FM diacetate (2 μM; Life Technologies, USA) for 20 minutes at 37° C. Following dye loading, ECs were again treated with L-NIO and NTG during a recovery phase for an additional 1 h in regular growth medium. EC culture media was then rinsed once with Krebs-Henseleit Buffer (KHB) containing 125 mM NaCl, 4.74 mM KCl, 2.5 mM CaCl₂, 1.2 mM KH₂PO₄ 1.2 mM MgSO₄, 5 mM NaHCO₃ and 10 mM Glucose (Sigma Aldrich, USA), replaced with fresh KHB and immediately subjected to live cell fluorescence imaging using Nikon Eclipse Ti microscope. At least 30 cells per condition were analyzed for total cell fluorescence using ImageJ software (NIH). For the second method, a Nitric Oxide Analyzer (NOA; Sievers, USA) was used to measure EC-secreted NO in culture medium. For this measurement, confluent ECs were treated overnight with either L-NIO (5 mM)±NTG (5 μM) or NTG alone, followed by sequential 1 h incubations with KHB without and with L-NIO±NTG at 37° C. KHB conditioned medium (4 replicates per condition) was then collected and analyzed using NOA.

ICAM-1 Clustering.

ECs were grown to confluence on glass coverslips under normal growth conditions and treated with L-NIO (5 mM)±[NTG (5 μM) or NTG-NL (5 μg/mL)] for 24 h. Next, a monocyte adhesion assay was performed (as described earlier) and the monocyte-EC co-cultures fixed, permeabilized with 0.1% Triton X-100, blocked with 2% bovine serum albumin (BSA; Millipore, USA), and sequentially incubated with primary anti-ICAM-1 mouse antibody (Santa Cruz Biotechnology, USA) and secondary FITC-conjugated DyLight 488 anti-mouse IgG (Vector Labs, USA). To visualize actin microfilaments, monocyte-EC co-cultures were incubated with Alexa Fluor 594-Phalloidin (BD Biosciences, USA). Coverslips were mounted onto glass slides and fluorescence images (15 per condition) were taken using a Nikon Eclipse Ti microscope fitted with a Nikon Digital Sight DS-Qi1Mc camera. ICAM-1 clustering index was determined using ImageJ by normalizing the total monocyte fluorescence (from at least 30 cells) to the background of the surrounding EC cytoplasm.

Detection of Endothelial Mitochondrial Superoxide Production.

ECs were plated at sub-confluence on gelatin-coated MatTek dishes under normal culture conditions and subjected to overnight treatment with either L-NIO (3 mM)±[NTG (5, 25, or 100 μM) or NTG-NL (5, 50 or 100 μg/mL)]. To detect mitochondrial superoxide, ECs were labeled with MitoSOX™ (Life Technologies, USA), a mitochondrial superoxide-sensitive fluorescent dye that is widely used to measure mitochondrial ROS production under various conditions, including NTG treatment. ECs were labeled with MitoSOX™ Red at a final dose of 5 μM in KHB for 10 minutes at 37° C., rinsed three times with KHB to remove excess dye and placed at 37° C. in KHB for an additional 10 minutes prior to live cell imaging. Fluorescence images (six per condition) were acquired using Nikon Eclipse Ti microscope and total cell fluorescence intensity from at least 20 cells was analyzed using ImageJ software.

Pulmonary Artery Ring Preparation and Isometric Tension Measurements.

All animal procedures were in accordance with the Animal Welfare Act, the Guiding Principles in the Care and Use of Animals approved by the Council of the American Physiological Society, the National Institutes of Health Guide for the Care and Use of Laboratory Animals, and Loma Linda University Institutional Animal Care and Use Committee (IACUC). Pregnant ewes were anesthetized with ketamine (10 mg/kg, IV) and midazolam (5 mg/kg, IV) and anesthesia maintained with inhalation of 1-3% isofluranein O₂ throughout surgery as required. Ewes and Fetuses were euthanized with an overdose of Euthasol (pentobarbital sodium, 100 mg/kg) and phenytoin sodium (10 mg/kg; Virbac, Ft. Worth, Tex.).

To assess arterial vasorelaxation in response to NTG treatment, pulmonary arteries (4^{th}-5^{th} order) were harvested from newborn fetal sheep (gestational period between 138-141 days), dissected free of parenchyma and cut into 5 mm long rings (at least 8 per condition) in ice-cold HEPES buffer (Sigma Aldrich, USA). Rings were then preincubated in L-NIO (1 mM)±[NTG (5 or 100 μM) or NTG-NL (5 or 100 μg/mL)] for 4 h at 37° C.; free NTG at 100 μM has previously been reported to induce NTG tolerance of isolated arterial rings within 4 h. Following treatment, rings were mounted onto tungsten wires under 0.5 g of resting tension in organ baths containing KHB, gassed with 95% O₂-5% CO₂, and maintained at 37° C. Arterial rings were rinsed once with KHB, allowed to equilibrate for at least 30 minutes, and re-tensioned prior to addition of 125 mM KCl (Sigma Aldrich, USA) to ensure rings were still functional. Rings were rinsed three times to remove KCl, allowed to relax, and preconstricted with 10 μM Serotonin (Tocris Bioscience, USA) to achieve 100% contraction. Rings were then exposed to increasing concentrations of free NTG (1 nM to 100 μM) and subsequent recordings of concentration-response curves were acquired using a force displacement transducer (AD Instruments, New Zealand) and analyzed using Prism (Graphpad Software Inc).

Statistics.

All data were obtained from multiple replicates (as described in the appropriate sections) and expressed as mean±standard error of mean (SEM). Statistical significance was determined using analysis of variance (ANOVA; InStat; Graphpad Software Inc.) followed by a Tukey multiple comparison post-hoc analysis. Results demonstrating significance were represented as *p<0.05, **p<0.01, or ***p<0.001.

NTG Exerts Anti-Inflammatory Effects on Activated ECs.

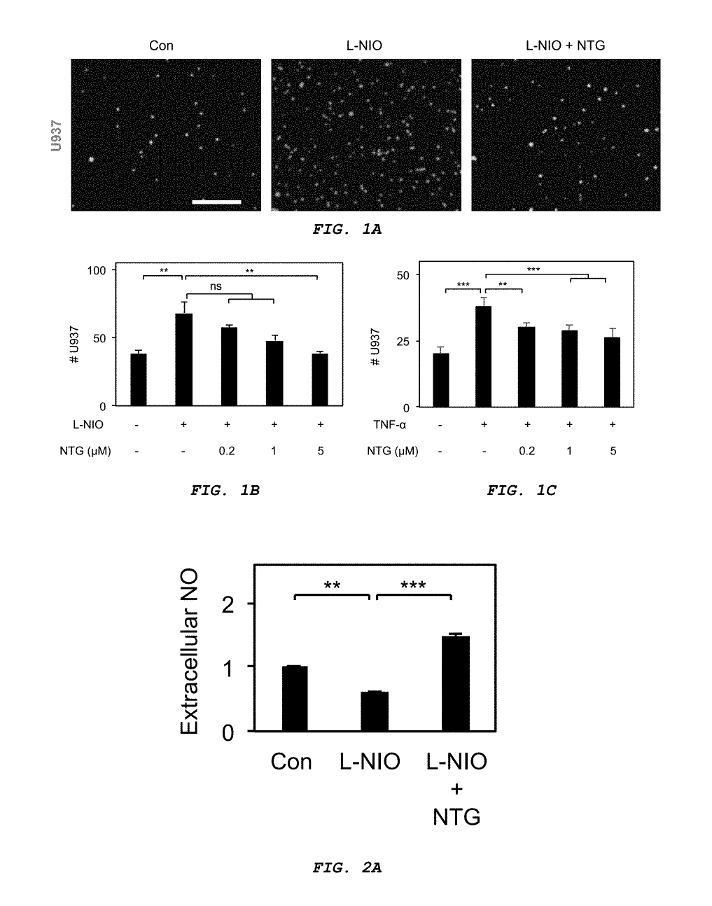

Since NTG enhances endothelial NO bioavailability through both spontaneous biotransformation and eNOS activation, it was questioned whether NTG could mimic the anti-inflammatory property of NO. To address this question, EC monolayers were treated with L-NIO (5 mM) or TNFα (10 ng/mL), which cause eNOS/NO deficiency and thereby enhance monocyte-EC adhesion both in vitro and in pathological conditions in vivo. Images of fluorescently-labeled adherent monocytes and their quantification revealed that addition of NTG to LNIO-treated ECs produced a dose-dependent inhibition of U937 monocytic cell adhesion to ECs (FIGS. 1A and 1B), with the inhibition being significant (p<0.01) at 5 μM dose where U937 cell adhesion was comparable with that on untreated ECs. Notably, NTG exerted a similar dose-dependent anti-inflammatory effect on cells activated with TNF-α (see FIG. 1C).

NTG Enhances Endothelial NO Production.

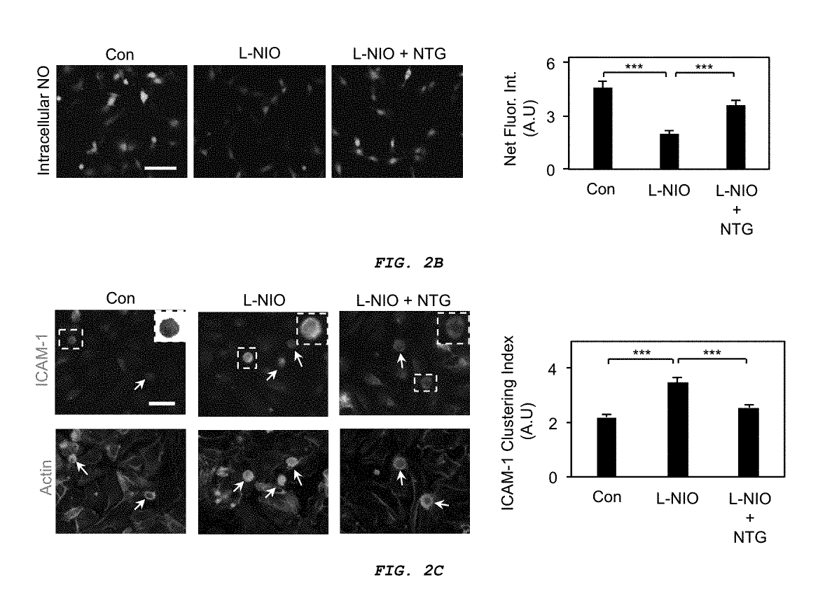

The potent and hitherto-unknown anti-inflammatory effect of NTG expectedly resulted from an increase in endothelial NO production, as confirmed by two independent approaches. Measurement of released (extracellular) NO by nitric oxide analyzer (NOA) revealed that addition of NTG to L-NIO-treated ECs produced a complete recovery of EC-dependent NO production (see FIG. 2A). These findings were independently confirmed by measurement of intracellular NO using a fluorescent NO-sensitive DAF-FM diacetate dye where quantification of fluorescence intensities revealed that NTG causes a significant (79%; p<0.001) recovery of NO in L-NIO-treated cells (see FIG. 2B).

NO is known to suppress leukocyte-EC adhesion by inhibiting the clustering and/or expression of endothelial cell adhesion molecules (CAMs). Quantitative analysis of fluorescent images of monocyte-EC co-cultures labeled with anti-ICAM-1 antibody and phalloidin (which stains actin cytoskeleton) revealed that NTG inhibited the significant increase (1.6-fold; p<0.001) in ICAM-1 clustering seen with NO-deficient (L-NIO-treated) cells (see FIG. 2C).

Synthesis and Physicochemical Characterization of Nanoliposomal NTG (NTG-NL).

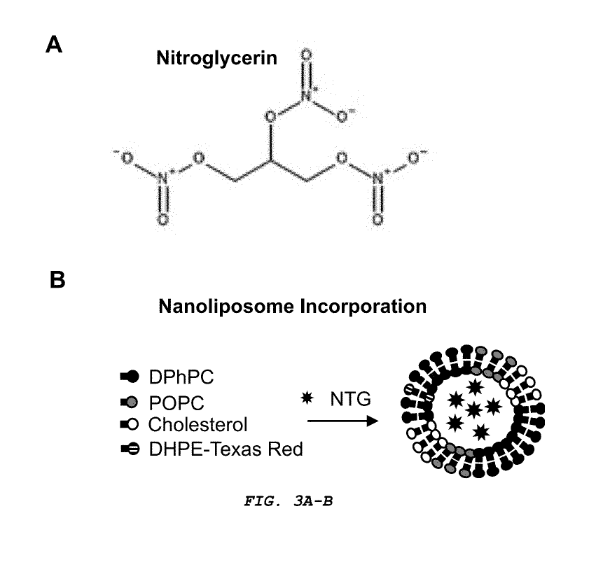

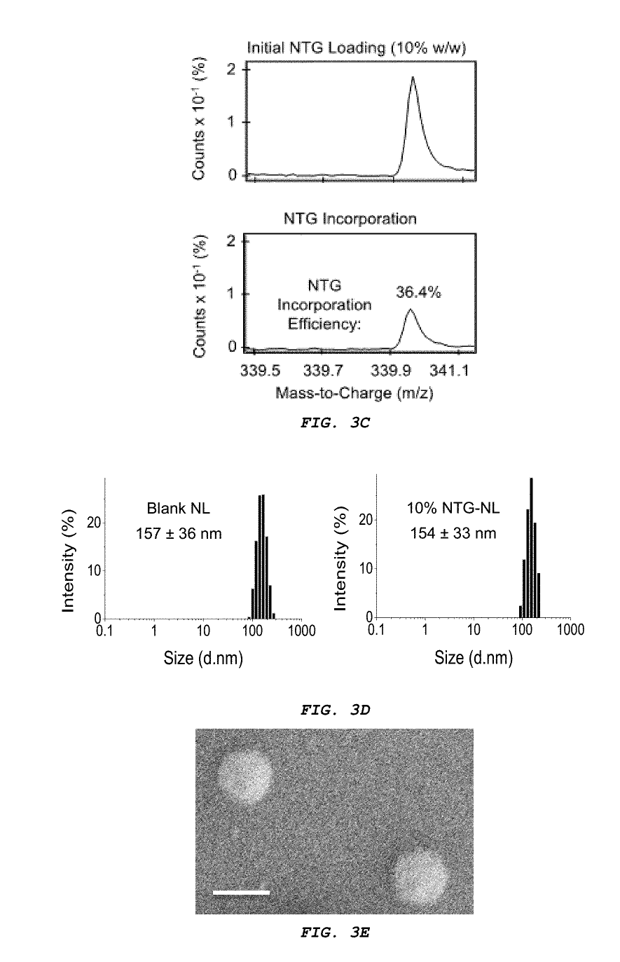

Despite important therapeutic implications of the anti-inflammatory effects of NTG, its systemic delivery using conventional high-dose formulations causes adverse effects in the form of enhanced mitochondrial superoxide production, thereby leading to NTG tolerance. To address this issue, the principles of nanotechnology were employed to incorporate NTG within nanoliposomes (NLs). NLs were made from a combination of four lipids (DPhPC, POPC, Cholesterol, and DHPE-Texas Red®. NL was chosen because the NTG molecule contains hydrophilic residues (see, e.g., FIG. 3A), which would facilitate its incorporation within the hydrophilic core of the nanoliposomes (see FIG. 3B).

Analysis of electrospray ionization-mass spectroscopy (ESI-MS) peaks revealed NTG uptake into nanoliposomes (NLs), which exhibited a dose-dependent increase in NTG loading (see FIG. 3C and Table 1).

TABLE 1
ESI-MS Analysis of NTG Incorportion Efficiency within Nanoliposomes
(NL)
NTG incorporation within NLs increased with increasing loading
(5, 10, and 25% wt. NTG/wt. NLs), although the maximum
incorporation efficiency was observed at the intermediate NTG
loading of 10% w/w (~37% incorporation efficiency). This trend is
consistent with drug loading within nanoparticles, as previously
reported.
ESI-MS Analysis of NTG Incorporation Efficiency within NLs.

| Initial NTG | | |
|---|---|---|
| Loading | Incorporated NTG | NTG Incorporation |
| (% w/w) | (Peak Area) | Efficiency (%) |
| 5 | 3039 | 15.6 |
| 10 | 14307 | 36.4 |
| 25 | 28653 | 23.4 |

These findings, which represent the first attempt to encapsulate NTG within a nanocarrier, identify NLs as the preferred vehicle for NTG packaging and delivery. Interestingly, although NTG incorporation within NLs understandably increased with increasing loading, the incorporation efficiency was highest (˜37% of initial NTG added) at an intermediate NTG loading of 10% (w/w) (see Table 1). Based on these findings, 10% (w/w) NTG-NL was chosen as the preferred nanoformulation for subsequent cell functional studies.

The size distribution profile of NLs, obtained using dynamic light scattering (DLS), revealed an average diameter of 157±36 nm and 154±33 nm for blank and NTG-NL, respectively (see FIG. 3D), which was independently confirmed by scanning electron microscopy (see FIG. 3E).

Cellular Uptake of Nanoliposomes.

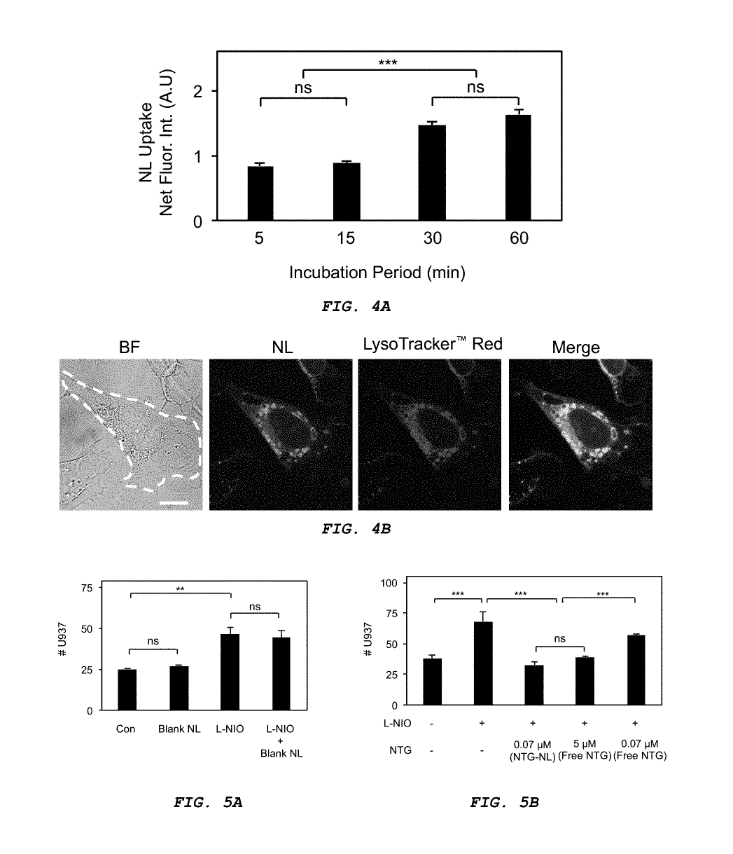

Following NL synthesis, their uptake was examined by cultured ECs. Fluorescein-loaded NLs (5 μg/mL) were added to ECs for 5, 15, 30, or 60 min prior to fixation and imaging. Quantification of intracellular fluorescence intensity measurements revealed that nanoliposomal uptake peaked at approximately 30 min, followed by a plateau between 30-60 min (see FIG. 4A). These internalized NLs expectedly localized within the acidic endocytic organelles viz. lysosomes and endosomes that line the perinuclear region (see FIG. 4B). Notably, although the amount of internalized NLs increased at higher NL doses, the percent internalization was highest (9%) at 5 μg/mL (see Table 2). Based on these observations of nanoliposomal uptake, all subsequent in vitro cell functional studies were performed following 30 min treatment with a 5 μg/mL dose of NL.

TABLE 2
NL Uptake by Cultured ECs.
When added to cultured ECs, fluorescein-containing nanoliposomes
undergo dose-dependent uptake by ECs, with the net internalized amount
increasing with increasing NL dose. However, this dose-dependent
increase in net NL uptake is inversely proportional to percent uptake by
ECs, which is the highest at 5 μg/mL dose and decreases progressively
with increasing NL dose.
Nanoliposome (NL) Uptake by Cultured ECs

| NL Conc. | NL Uptake | % NL |
|---|---|---|
| (μg/ml) | Net Fluor. Int. (A.U) | Uptake |
| 5 | 72 | 9.0 |
| 10 | 163 | 5.1 |
| 50 | 579 | 2.0 |
| 100 | 1580 | 0.8 |

NTG-NL Exerts Superior Anti-inflammatory Effects.

For NTG-NL to be truly effective as an anti-inflammatory therapy, it is important that blank NLs exert no inflammatory effects. To confirm this, monocyte adhesion to ECs treated with blank NLs was analyzed. Quantification of adherent U937 cells revealed that monocyte adhesion to blank NL-treated ECs is comparable to that seen on untreated ECs (see FIG. 5A). Further, treatment of ECs with blank NLs failed to suppress L-NIO-induced increase in U937 cell-EC adhesion (see FIG. 5A). These data clearly indicate that blank NLs are totally inert to ECs.

Nanoparticles enhance drug efficacy by simultaneously increasing drug half-life and facilitating rapid cellular uptake. Thus, it was questioned whether the internalized NTG-NLs could improve the anti-inflammatory effect of incorporated NTG. The studies presented herein indicate that addition of NTG-NL (5 μg/mL) to L-NIO-treated ECs produced a significant inhibition (52%; p<0.001) of U937 cell adhesion to ECs, with the number of adherent monocytes returning to the levels seen on untreated ECs (see FIG. 5B). This reduction in monocyte-EC adhesion by NTG-NL was comparable to the anti-inflammatory effect produced by a 5 μM dose of free NTG. Determination of the total amount of NTG delivered through nanoliposomal formulation revealed that NTG-NL produced its potent anti-inflammatory effect at 0.07 μM, which is 70-fold less than the effective free NTG dose (5 μM). In other words, NTG-NL was found to be 70-fold more effective than free NTG in suppressing endothelial activation. That this remarkable increase in therapeutic efficacy resulted primarily from nanoformulation of NTG was confirmed by the observation that an equivalent amount (0.07 μM) of free NTG failed to produce a significant anti-inflammatory effect (see FIG. 5B).

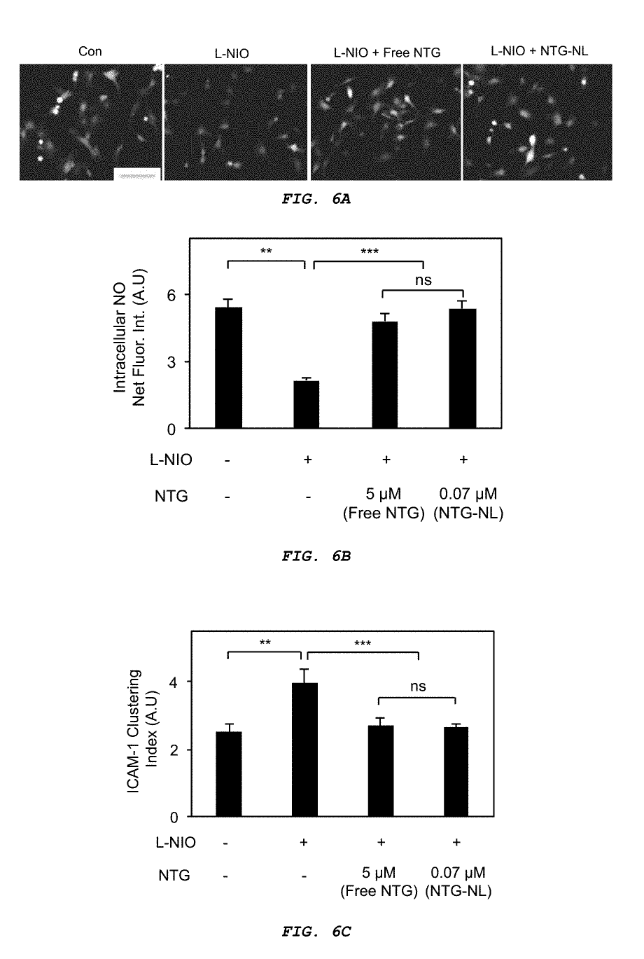

Further, similar to free NTG, the anti-inflammatory effect of NTG-NL correlated strongly with its ability to enhance endothelial NO production (see FIG. 6A-B) and suppress ICAM-1 clustering (see FIG. 6C).

NTG-NL Prevents Endothelial Superoxide Formation Associated with High NTG Dose.

To further underscore the superior therapeutic efficacy of NTG-NL that leads to significant reduction of effective NTG dose, its ability to prevent excessive mitochondrial superoxide formation associated with high doses of free NTG was examined. Mitochondrial superoxide is a reactive oxygen specie (ROS) that is formed as a byproduct during NTG bioconversion to NO. Importantly, mitochondrial superoxide inhibits the activity of mitochondrial aldehyde dehydrogenase (ALDH-2), the chief enzyme responsible for NTG bioconversion. Thus, excessive amounts of mitochondrial superoxide generated in response to clinically-administered high NTG doses significantly impairs ALDH-2 activity and NTG bioconversion, thereby leading to the onset of clinical NTG tolerance. As such, levels of mitochondrial superoxide are used as a reliable marker for NTG tolerance. Since NTG-NL reduced the effective therapeutic NTG dose by approximately two folds, it was examined to see whether a similar fold increase in NTG-NL and free NTG doses exerts differential effects on mitochondrial superoxide formation.

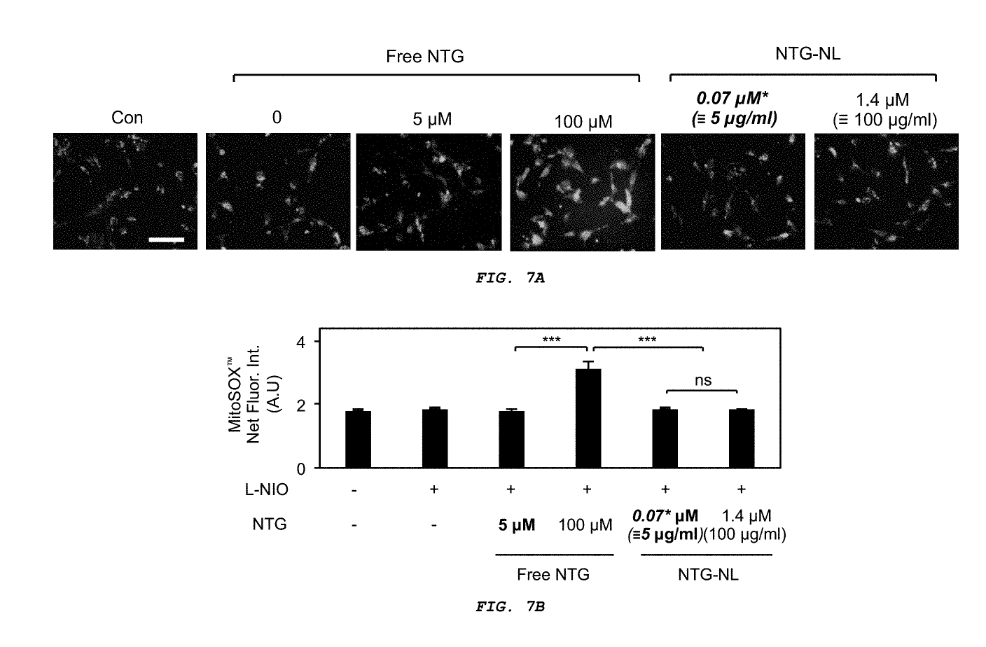

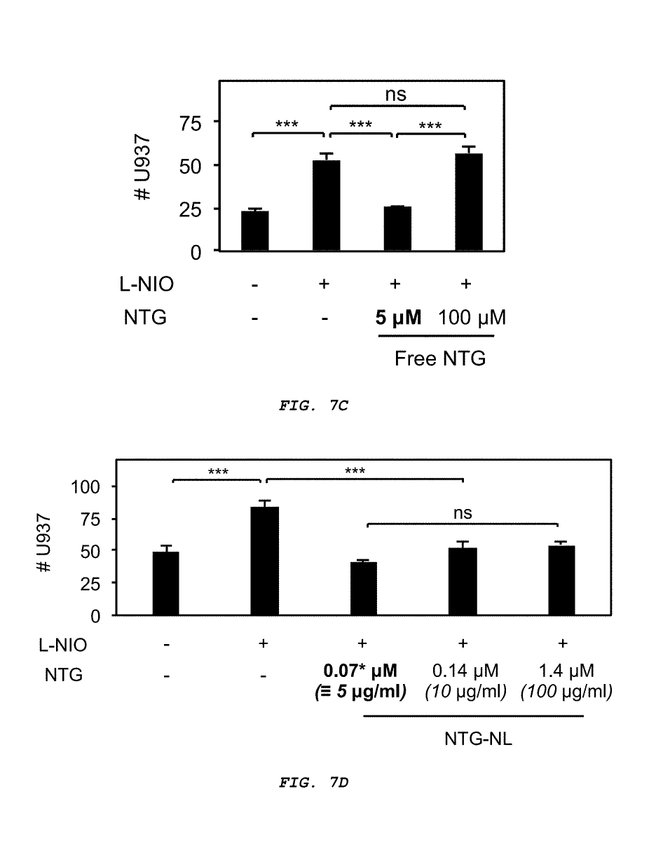

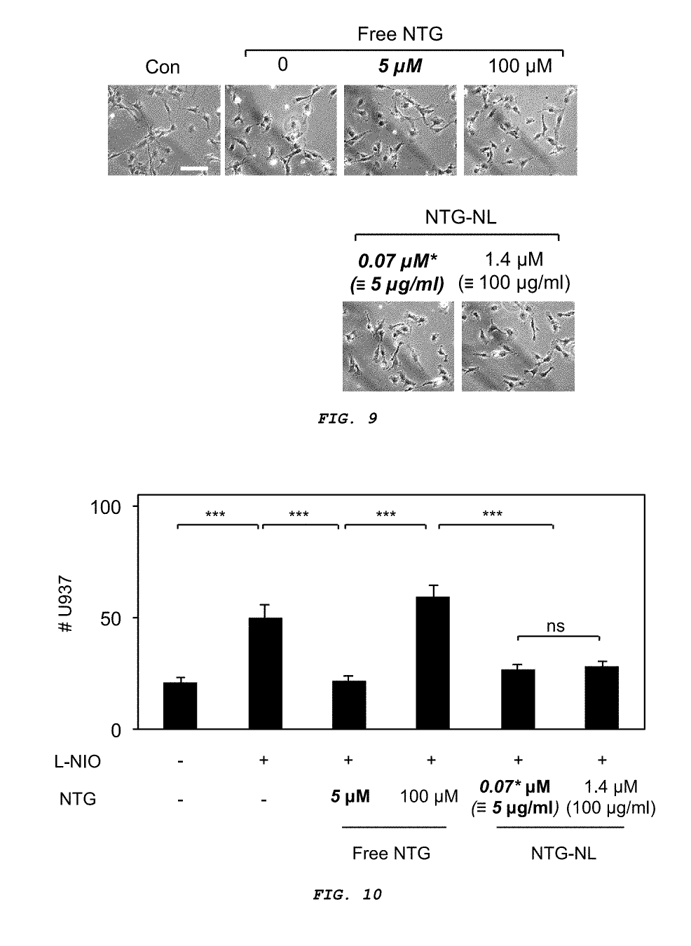

To detect mitochondrial superoxide in NTG-treated ECs, cells were labelled with MitoSOX™, a mitochondrial superoxide-sensitive fluorescent dye that is used to measure mitochondrial ROS production under various conditions, including NTG treatment. Quantitative analysis of fluorescent intensity of MitoSOX™-labeled ECs revealed that cells treated with a free NTG dose 20-fold higher than its effective anti-inflammatory dose of 5 μM produced a 2-fold (p<0.001) increase in mitochondrial superoxide formation (see FIGS. 7A and 7B). As shown in FIG. 9, this difference in MitoSOX™ fluorescence intensity is a true reflection of the differences in mitochondrial superoxide production and not an artifact resulting from varying cell density or spreading. More importantly, however, NTG-NL did not elicit any increase in mitochondrial superoxide production when used at a similar 20-fold higher dose (i.e., at 100 μg/mL).

Consistent with the view that excessive mitochondrial superoxide production at high NTG doses inhibits ALDH-2 activity and, thus, NTG bioconversion to NO, a drastic loss (˜2-fold decrease; p<0.001) in the anti-inflammatory effects of free NTG was observed at 100 μM (see FIG. 7C). In contrast, NTG-NL retained its potent immunosuppressive effects (84% inhibition; p<0.001) when used at the 20-fold higher dose of 100 μg/mL (see FIG. 7D).

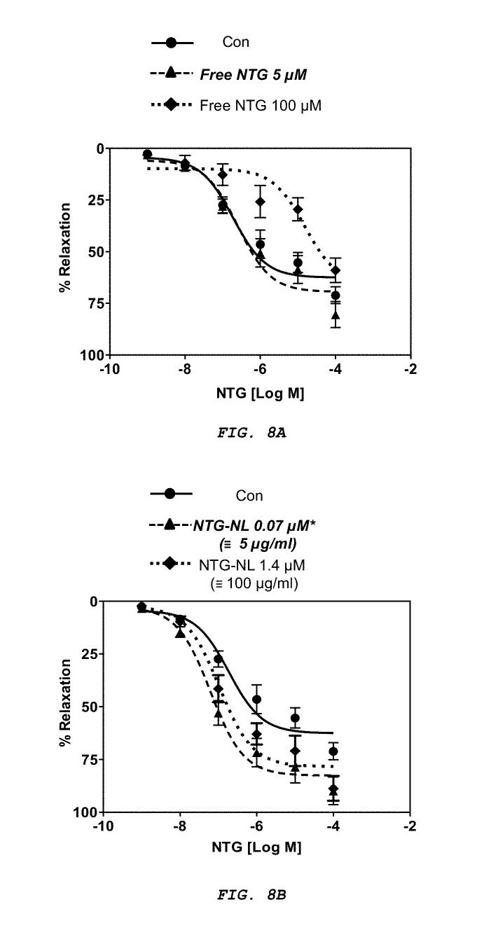

Another important effect of excessive mitochondrial superoxide production and associated reduction of NTG bioconversion at high NTG doses is impaired vasodilatory response to acute NTG treatment (tolerance). Since the high NTG-NL dose of 100 μg/mL did not enhance mitochondrial superoxide formation, it was hypothesized that high-dose NTG-NL treatment will also exert no inhibitory effect on acute NTG-dependent vasodilation. To test this hypothesis, an ex vivo arterial vasorelaxation assay was performed. This assay is used to recapitulate NTG-induced vasorelaxation and tolerance observed in vivo. Isolated sheep pulmonary arteries were pretreated with free NTG (5 and 100 μM) or NTG-NL (5 and 100 μg/mL) for 4 h, followed by measurement of NTG-induced vasorelaxation. The 4 h pretreatment was sufficient for arterial ECs to uptake NTG-NL and release NO, as indicated by the significant inhibition in monocyte adhesion to NTG-NL-treated ECs (See FIG. 10). Further, and more importantly, while pulmonary arteries pretreated with 100 μM free NTG exhibited a significant rightward shift in NTG concentration-response curve (IC₅₀ increasing from 0.2 to 15 μM; FIG. 8A and Table 3), NTG-NL-treated arteries maintained their normal vasodilatory responsiveness at both 5 and 100 μg/mL doses (see FIG. 8B and Table 3).

TABLE 3
Effects of Free NTG and NTG-NL Treatment on IC₅₀ and
Maximal Relaxations in Isolated Pulmonary Sheep Arteries.
Pulmonary sheep arteries pretreated with the effective (anti-inflammatory)
free NTG dose of 5 μM exhibit normal NTG dose-relaxation profile
similar to untreated controls. In contrast, arteries pretreated
with a 20-fold higher free NTG dose (100 μM) demonstrate a
significant increase in mean IC₅₀ values, which is indicative of NTG
tolerance. Remarkably, pretreatment with NTG-NL at both 5 and
100 μg/mL doses exhibit no evidence of NTG tolerance. IC₅₀ are
concentrations that produced 50% relaxation in response to NTG
stimulation. The maximal relaxation response was, however, similar
in the control and both free NTG-treated arteries while the NTG-NL-
treated arteries exhibited marginally improved maximal relaxation
response. The precise reason for this improvement in maximal
relaxation by NTG-NL remains unclear. **, p < 0.01; ***, p < 0.001.
Therapeutic dose is highlighted in bold. Data are expressed as mean ±
SEM.
Effects of Free NTG and NTG-NL Treatment on IC₅₀ and
Maximal Relaxation in Isolated Pulmonary Arteries.

| | | Maximal Relaxation |
|---|---|---|
| | −Log IC₅₀ (Mean IC₅₀, μM) | (%) |
| Con | 6.73 ± 0.14 (0.19) | 63 ± 3 |
| Free NTG 5 μM | 6.60 ± 0.18 (0.25) | 69 ± 4* |
| Free NTG 100 μM | 4.81 ± 0.24*** (15.5) | 66 ± 9 |
| NTG-NL 0.06 μM* | 7.21 ± 0.15 (0.06) | 83 ± 3 |
| (≡5 μg/ml) | | |
| NTG-NL 1.2 μM | 6.98 ± 0.15 (0.10) | 78 ± 4 |
| (≡100 μg/ml) | | |

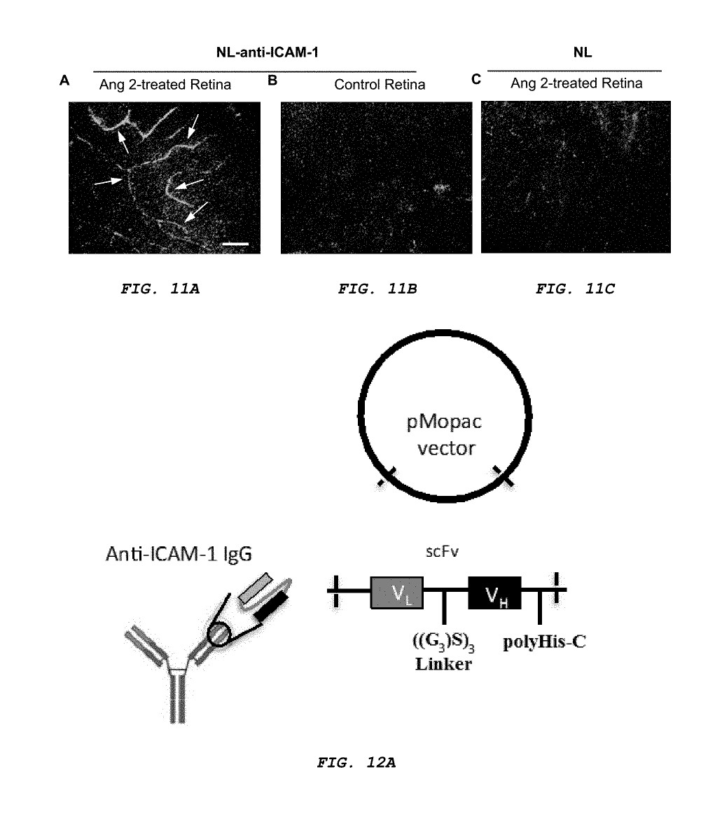

To demonstrate in vivo targeting of anti-ICAM-1-modified NLs, mouse retinal vessels were inflamed using a pro-inflammatory cytokine, angiopoietin-2 (ANG-2), a known enhancer of endothelial ICAM-1 expression. Intravenous injection of both modified and unmodified NLs (containing Texas Red™-labeled lipids to facilitate imaging) followed by fluorescence imaging reveal that anti-ICAM-1-modified NLs preferentially accumulate within ICAM-1-expressing (ANG-2-treated) retinal vessels (see, FIGS. 11A and B). Importantly, this ICAM-1-targeting by NLs was not observed with unmodified NLs (see, FIG. 11C).

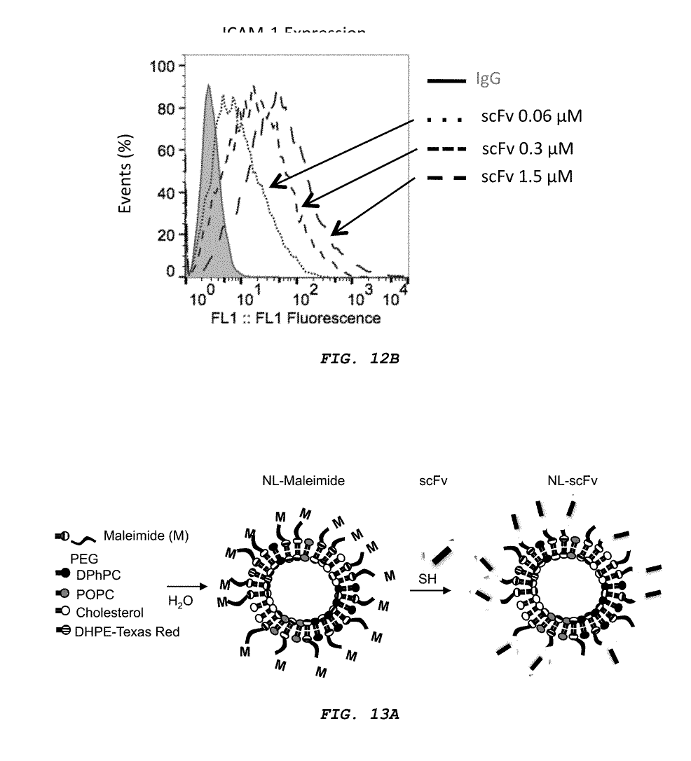

Whole ICAM-1 antibodies contain an Fc domain, which causes endogenous complement activation and subsequent clearance of injected ICAM-1 antibodies by a body's immune cells. To minimize or eliminate this effect, engineering of shorter fragments of anti-ICAM-1, specifically the single-chain variable fragment (scFv), was explored to further enhance NL accumulation at sites of vascular inflammation (ICAM-1 expression). Anti-ICAM-1 scFv was engineered using previously established variable Light and Heavy chain amino acid sequences with a conventional (Gly₃Ser)₄ linker, and a reactive cysteine at the heavy chain terminus that would facilitate Maleimide-thiol surface chemistries (see, FIG. 12A). To demonstrate the ability of scFv to bind human ECs, scFv was added to TNF-α-stimulated (ICAM-1-expressing) ECs. Flow cytometry measurements revealed that anti-ICAM-1 scFv bound activated ECs in a dose-dependent manner (see, FIG. 12B).

As a building block for these NLs, a combination of five lipids (DSPE-PEG₂₀₀₀-Maleimide, DPhPC, POPC, Cholesterol, and DHPE-Texas Red) were used, as described herein (see, FIG. 13A). Specifically, to render NLs suitable for ICAM-1 targeting, DSPE-PEG₂₀₀₀-Maleimide was incorporated within the lipid bilayer, which serves two purposes: firstly, the Maleimide (M) group will permit chemical conjugation of anti-ICAM-1 scFv onto NL surface, and secondly, poly (ethylene glycol) (PEG₂₀₀₀) chains will provide “stealth” to the NLs so it can avoid capture by circulating immune cells and the reticuloendothelial (RES) systems of liver and spleen.

To achieve successful NL-scFv conjugation, the conventional Maleimide-Thiol chemistry was employed to covalently conjugate thiol-functionalized scFv to DSPE-PEG-Maleimide. scFv conjugation to NL surface was detected using antibody labeling and subsequent fluorescence intensity measurement (using a Flexstation 2 fluorescent microplate reader) corresponding to the binding of fluorophore-labeled secondary antibody. Representative line graph shows the linear correlation between the amount of scFv added to NL suspension (x-axis) and the amount conjugated to NL surface (fluorescence intensity; y-axis) (n=3 per condition) (see, FIG. 13B).

To demonstrate the therapeutic potential of this scFv-modified NLs (NL-scFv), firstly, the ICAM-1-targeting potential of NL-scFv was evaluated in TNF-α-stimulated (ICAM-1-expressing) human pulmonary arterial ECs (PAECs). These cells were chosen because they are a crucial therapeutic target in pulmonary arterial hypertension (PAH), a chronic, debilitating, and intractable condition that is characterized by significant ICAM-1-expression on PAECs, and subsequent pulmonary arterial inflammation. PAH is also marked by chronic vasoconstriction. Notably, PAECs are known to express ICAM-1 at ˜30-fold greater density than ECs in vessels of other organs. To demonstrate the capability of NL-scFv conjugates to preferentially target ICAM-1-expressing PAECs, fluorescently-labeled NL-scFv was added to both untreated (no TNF-α) and TNF-α-stimulated PAECs. Quantitative measurement of fluorescence intensity of NL-treated ECs revealed a six-fold (p<0.001) greater binding of NL-scFv to stimulated PAECs when compared with untreated control (see, FIG. 14) at the saturation dose of 1.9 μM. This data demonstrates the potent and selective ICAM-1-targeting capability of these NLs and their potential use as a therapeutic carrier for targeted delivery of PAH therapies to the lungs.

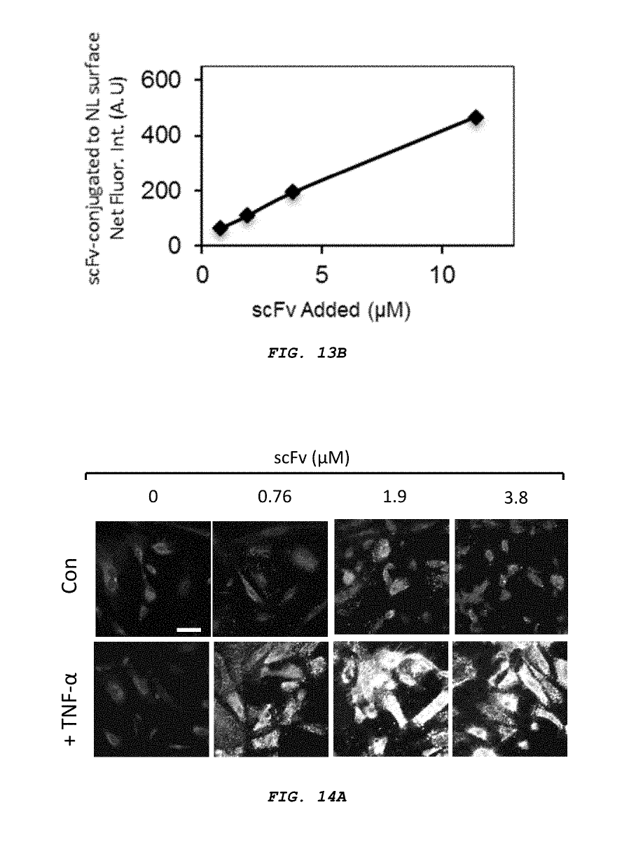

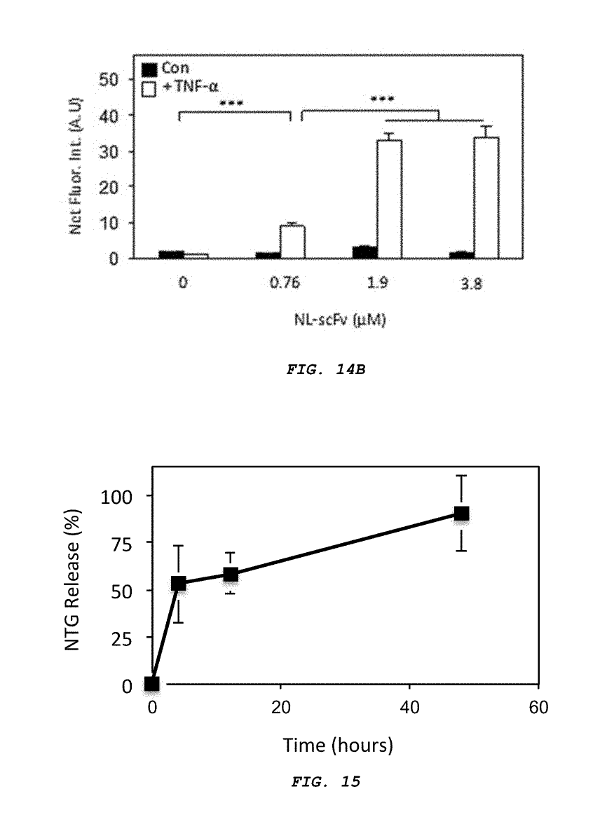

To render these site-targeting NLs useful for therapeutic applications, NTG, which has been demonstrated to exhibit potent anti-inflammatory effects, was incorporated into NLs at 10% (w/w) ratio, and its release kinetics were evaluated over a 48 hr time period. ESI-MS quantitative measurements revealed an initial burst release of NTG lasting approximately 12 hr, followed by a steady release over 48 hr, with approximately ˜90% of the incorporated NTG being released over 48 hr (see, FIG. 15).

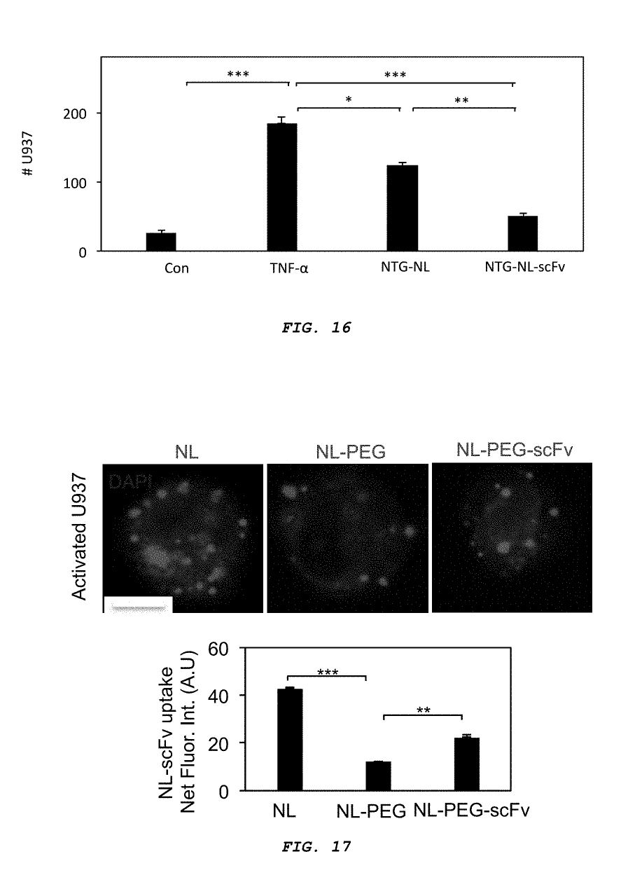

To demonstrate the anti-inflammatory potential of these ICAM-1-targeting NLs for PAH treatment, NTG was incorporated within the NLs with or without scFv functionalization. Next, the ability of NTG-loaded NLs or NL-scFvs to inhibit U937 cell adhesion to TNF-α-stimulated ECs was examined using an in vitro U937 cell-EC adhesion assay. TNF-α-treated PAECs were exposed to NTG-NL or NTG-NL-scFv for 4 hr prior to addition of fluorescently-labeled U937 cells for 30 min. Quantification of adherent U937 cells revealed that, when compared with PAECs treated with NTG-NL, those treated with NTG-NL-scFv exhibited a two-fold lower (p<0.01) adhesion of U937 cells (see, FIG. 16). This increased anti-inflammatory effect produced by NTG-NL-scFv likely reflects the greater uptake of NTG-NL-scFV by ICAM-1-expressing PAECs.

A major limitation of NLs is that upon injection into the bloodstream, they are readily taken up by circulating immune cells and readily cleared from blood circulation, which reduces NL site-targeting potential and subsequent therapeutic effects. To address this issue, PEGylated lipids were used to synthesize the NLs as the hydrophilicity of PEGs minimizes protein adsorption and thus provides “stealth” from the immune cells. To confirm this, non-PEGylated and PEGylated NLs were incubated with activated monocytes (macrophage-like). When compared with non-PEGylated NLs, both PEG- and PEG-scFv functionalized NLs exhibited significantly (p<0.001) lower degree of internalization by activated U937 monocytes (see, FIG. 17). Notably, there was a small but significant reduction in the stealth capability of NL-PEG-scFv (when compared with NL-PEG alone), which may be attributed to steric hindrance caused by the relatively large scFv (˜26 kDa) when compared with the smaller PEG chains (2 kDa).

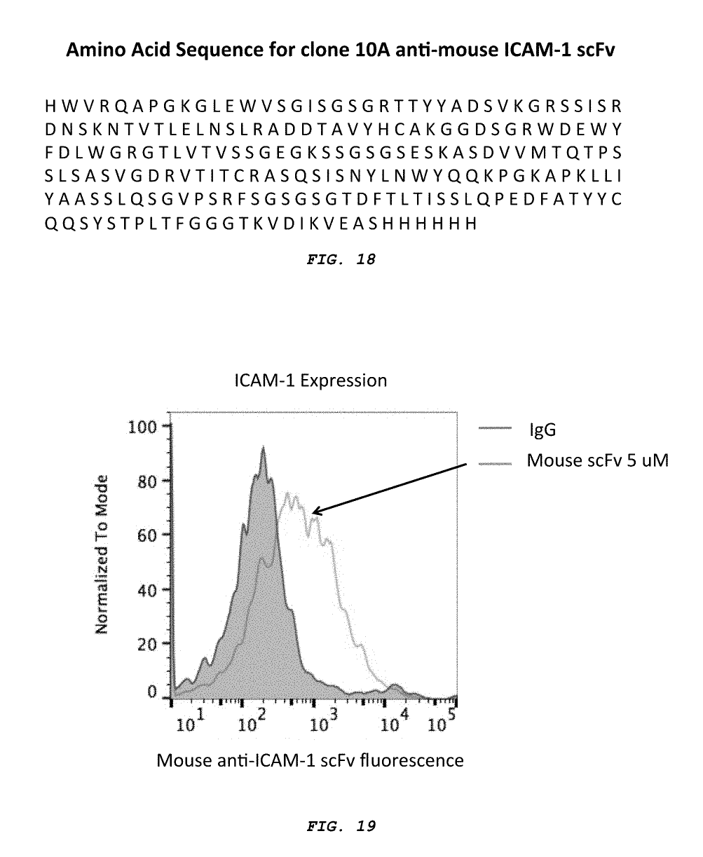

To address the need to evaluate the translational potential of scFv-modified nanoliposomal NTG in preclinical models of vascular inflammation, unique scFv sequences targeting ‘mouse’ ICAM-1 were identified by screening a proprietary naïve scFv phage library against recombinant mouse ICAM-1 antigen. The amino acid sequence of the variable Light and Heavy chains of one scFv clone (clone 10A) is shown in FIG. 18.

To demonstrate the ability of mouse scFv to bind ICAM-1-expressing mouse ECs, scFv (5 μM) was added to activated (TNF-α-stimulated) ECs. Flow cytometry measurements revealed that the anti-ICAM-1 scFv binds significantly to ICAM-1 expressed on mouse ECs (see, FIG. 19). Based on this data, it can be hypothesized that NLs modified with this scFv sequence will selectively bind ICAM-1-expressing mouse vessels after intravenous injection.

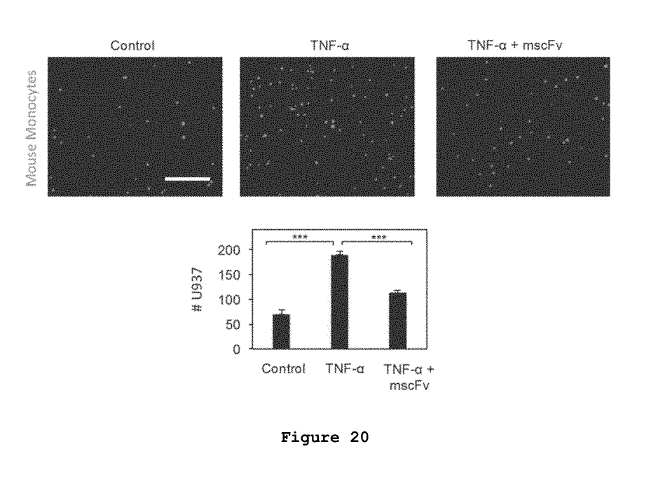

Since scFv clone 10A binds mouse ICAM-1, which is required for immune cell binding, it is possible that the scFv also exhibits ICAM-1 function blocking effects. To examine this, mouse ECs were inflamed using TNF-α, followed by treatment with scFv (5 μM) and subsequent incubation of fluorescently-labeled mouse monocytes. Representative fluorescent images of adherent mouse monocytes and quantification of monocyte count reveals a significant (p<0.001) reduction in monocyte binding to ECs treated with scFv than to untreated ECs (see, FIG. 20).

A number of embodiments have been described. Nevertheless, it will be understood that various modifications may be made without departing from the spirit and scope of the description. Accordingly, other embodiments are within the scope of the following claims.

1. A nitroglycerin-nanoliposome (NTG-NL) formulation comprising: nitroglycerin incorporated in nanoliposomes made from a plurality of lipids, wherein the nanoliposomes have a diameter between 10 to 500 nm.

2. The NTG-NL formulation of claim 1, wherein the nanoliposomes are unilamellar liposomes or micelles.

3. The NTG-NL formulation of claim 1, wherien the nanoliposomes are multilamellar liposomes.

4. The NTG-NL formulation of claim 1, wherein the plurality of lipids comprise phospholipids or derivatives thereof selected from phosphatidylcholine, phosphatidic acid, phosphatidylethanolamine, phosphatidylglycerol, phosphatidylserine, lysophosphatidylcholine, and/or any derivative thereof.

5. The NTG-NL formulation of claim 4, wherein the phospholipid derivatives are selected from 1,2-di-(3,7,11,15-tetramethylhexadecanoyl)-sn-glycero-3-phosphocholine, 1,2-didecanoyl-sn-glycero-3-phosphocholine, 1,2-dierucoyl-sn-glycero-3-phosphate, 1,2-dierucoyl-sn-glycero-3-phosphocholine, 1,2-dierucoyl-sn-glycero-3-phosphoethanolamine, 1,2-dilinoleoyl-sn-glycero-3-phosphocholine, 1,2-dilauroyl-sn-glycero-3-phosphate, 1,2-dilauroyl-sn-glycero-3-phosphocholine, 1,2-dilauroyl-sn-glycero-3-phosphoethanolamine, 1,2-dilauroyl-sn-glycero-3-phospho-(1′-rac-glycerol), 1,2-dimyristoyl-sn-glycero-3-phosphate, 1,2-dimyristoyl-sn-glycero-3-phosphocholine, 1,2-dimyristoyl-sn-glycero-3-phosphoethanolamine, 1,2-dimyristoyl-sn-glycero-3-phosphoglycerol, 1,2-dimyristoyl-sn-glycero-3-phosphoserine, 1,2-dioleoyl-sn-glycero-3-phosphate, 1,2-dioleoyl-sn-glycero-3-phosphocholine, 1,2-dioleoyl-sn-glycero-3-phosphoethanolamine, L-alpha-phosphatidyl-DL-glycerol, 1,2-dioleoyl-sn-glycero-3-phosphoserine, 1,2-dipalmitoyl-sn-glycero-3-phosphate, 1,2-dipalmitoyl-sn-glycero-3-phosphocholine, 1,2-dipalmitoyl-sn-glycero-3-phosphoethanolamine, 1,2-dipalmitoyl-sn-glycero-3-phosphoglycerol, 1,2-dipalmitoyl-sn-glycero-3-phosphoserine, 1,2-distearoyl-sn-glycero-3-phosphate, 1,2-distearoyl-sn-glycero-3-phosphocholine, 1,2-distearoyl-sn-glycero-3-phosphoethanolamine, 1,2-distearoyl-sn-glycero-3-phosphoglycerol, egg sphingomyelin, egg-PC, hydrogenated Egg PC, hydrogenated Soy PC, 1-myristoyl-sn-glycero-3-phosphocholine, 1-palmitoyl-sn-glycero-3-phosphocholine, 1-stearoyl-sn-glycero-3-phosphocholine, 1-myristoyl-2-palmitoyl-sn-glycero 3-phosphocholine, 1-myristoyl-2-stearoyl-sn-glycero-3-phosphocholine, 1-palmitoyl-2-myristoyl-sn-glycero-3-phosphocholine, 1-palmitoyl-2-oleoyl-sn-glycero-3-phosphocholine, 1-palmitoyl-2-oleoyl-sn-glycero-3-phosphoethanolamine, 1-palmitoyl-2-oleoyl-sn-glycero-3-phosphoglycerol, 1-palmitoyl-2-stearoyl-sn-glycero-3-phosphocholine, 1-stearoyl-2-myristoyl-sn-glycero-3-phosphocholine, 1-stearoyl-2-oleoyl-sn-glycero-3-phosphocholine, and/or 1-stearoyl-2-palmitoyl-sn-glycero-3-phosphocholine.

6. The NTG-NL formulation of claim 1, wherein the nanoliposome further comprises one or more of cholesterol, polyethylene glycol and/or site-targeting moeities.

7-8. (canceled)

9. The NTG-NL formulation of claim 6, where the one or more site-targeting moieties is selected from the group consisting of a peptide, an aptamer, an antibody, and antibody fragment.

10. The NTG-NL formulation of claim 9, where the antibody fragment is selected from the group consisting of F(ab′)₂, Fab, Fab′ and scFv.

11. The NTG-NL formulation of claim 1, wherein the NTG-NL formulation is formulated for enteral delivery, parenteral delivery, topical delivery, or by inhalation.

12. The NTG-NL formulation of claim 1, wherein the nitroglycerin to nanoliposome ratio by weight is from 1:20 to 20:1.

13. (canceled)

14. The NTG-NL formulation of claim 12, wherein the nitroglycerin to nanoliposome ratio is 1:10.

15. The NTG-NL formulation of claim 1, wherein at least a portion of the plurality of lipids are conjugated with polyethylene glycol (PEG).

16. The NTG-NL formulation of claim 15, wherein a portion of the PEG-conjugated lipids further comprise maleimide groups.

17. The NTG-NL formulation of claim 15, wherein at least a portion of the PEG-conjugated lipids further comprise a site-targeting moiety.

18. The NTG-NL formulation of claim 17, wherein the site-targeting moiety is conjugated to the PEG-conjugated lipids using maleimide-thiol reaction chemistry.

19-21. (canceled)

22. The NTG-NL formulation of claim 15, wherein the nitroglycerin to nanoliposome ratio by weight is from 1:20 to 20:1.

23. (canceled)

24. The NTG-NL formulation of claim 22, wherein the nitroglycerin to nanoliposome ratio is 1:10.

25. A method for treating a disease or disorder associated with vascular inflammation, hyperpermeability, regression or vasoconstriction; loss of endogenous vascular endothelial nitric oxide; increased expression of endothelial cell adhesion molecules; or increased clustering of endothelial cell adhesion molecules in a subject comprising administering the NTG-NL formulation of claim 1 to the subject.

26. The method of claim 25, wherein the disease or disorder associated with vascular inflammation, hyperpermeability, regression or vasoconstriction; loss of endogenous vascular endothelial nitric oxide; increased expression of endothelial cell adhesion molecules; or increased clustering of endothelial cell adhesion molecules is selected from pulmonary arterial hypertension (PAH), atherosclerosis, diabetic vascular complications, asthma, chronic peptic ulcer, tuberculosis, rheumatoid arthritis, chronic periodontitis, ulcerative colitis, Crohn's disease, chronic sinusitis, and chronic active hepatitis.

27. The method of claim 26, wherein the diabetic vascular complication is selected from the group consisting of retinopathy, nephropathy, neuropathy, and cardiovascular disease.

28. (canceled)

29. A method for treating a disease or disorder associated with vascular inflammation, hyperpermeability, regression or vasoconstriction; loss of endogenous vascular endothelial nitric oxide; increased expression of endothelial cell adhesion molecules; or increased clustering of endothelial cell adhesion molecules in a subject comprising administering the NTG-NL formulation of claim 15 to the subject.

30. The method of claim 29, wherein the disease or disorder associated with vascular inflammation, hyperpermeability, regression or vasoconstriction; loss of endogenous vascular endothelial nitric oxide; increased expression of endothelial cell adhesion molecules; or increased clustering of endothelial cell adhesion molecules is selected from the group consisting of pulmonary arterial hypertension (PAH), atherosclerosis, diabetic vascular complications, asthma, chronic peptic ulcer, tuberculosis, rheumatoid arthritis, chronic periodontitis, ulcerative colitis, Crohn's disease, chronic sinusitis, and chronic active hepatitis.

31. The method of claim 30, wherein the disease or disorder is pulmonary arterial hypertension (PAH) or atherosclerosis.
